SEQUENCE LISTING (60 sequences)

SEQ ID NO: 1 - protein, 8 aa, synthetic construct
GFTFSDYA

SEQ ID NO: 2 - protein, 8 aa, synthetic construct
ISSTGSYK

SEQ ID NO: 3 - protein, 12 aa, synthetic construct
LASVITNDYFEY

SEQ ID NO: 4 - protein, 7 aa, synthetic construct
GFTFSDY

SEQ ID NO: 5 - protein, 6 aa, synthetic construct
SSTGSY

SEQ ID NO: 6 - protein, 10 aa, synthetic construct
SVITNDYFEY

SEQ ID NO: 7 - protein, 5 aa, synthetic construct
DYAMS

SEQ ID NO: 8 - protein, 17 aa, synthetic construct
TISSTGSYKYYLDSVKG

SEQ ID NO: 9 - protein, 119 aa, synthetic construct
EVQLVESGGGLVKPGGSLKLSCAASGFTFSDYAMSWFRQAPEKRLEWVATISSTGSYKYYLDSVKGRFTISRDNSKNTLYLQMSSLRSEDTAVYYCLASVITNDYFEYWGQGTLVTVSS

SEQ ID NO: 10 - protein, 449 aa, synthetic construct
EVQLVESGGGLVKPGGSLKLSCAASGFTFSDYAMSWFRQAPEKRLEWVATISSTGSYKYYLDSVKGRFTISRDNSKNTLYLQMSSLRSEDTAVYYCLASVITNDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 6 aa, synthetic construct
QDVGTA

SEQ ID NO: 12 - skipped

SEQ ID NO: 13 - protein, 9 aa, synthetic construct
QQYANLPYT

SEQ ID NO: 14 - protein, 11 aa, synthetic construct
RASQDVGTAVA

SEQ ID NO: 15 - protein, 7 aa, synthetic construct
WASTRHT

SEQ ID NO: 16 - protein, 107 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYANLPYTFGGGTKLEIK

SEQ ID NO: 17 - protein, 214 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYANLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 8 aa, synthetic construct
GFTFSDYA

SEQ ID NO: 19 - protein, 8 aa, synthetic construct
ISSTGSYS

SEQ ID NO: 20 - protein, 12 aa, synthetic construct
LASSYTNDYFEY

SEQ ID NO: 21 - protein, 7 aa, synthetic construct
GFTFSDY

SEQ ID NO: 22 - protein, 6 aa, synthetic construct
SSTGSY

SEQ ID NO: 23 - protein, 10 aa, synthetic construct
SSYTNDYFEY

SEQ ID NO: 24 - protein, 5 aa, synthetic construct
DYAMS

SEQ ID NO: 25 - protein, 17 aa, synthetic construct
TISSTGSYSYYLDSVKG

SEQ ID NO: 26 - protein, 119 aa, synthetic construct
EVQLVESGGGLVKPGGSLKLSCAASGFTFSDYAMSWFRQAPEKRLEWVATISSTGSYSYYLDSVKGRFTISRDNSKNTLYLQMSSLRSEDTAVYYCLASSYTNDYFEYWGQGTLVTVSS

SEQ ID NO: 27 - protein, 449 aa, synthetic construct
EVQLVESGGGLVKPGGSLKLSCAASGFTFSDYAMSWFRQAPEKRLEWVATISSTGSYSYYLDSVKGRFTISRDNSKNTLYLQMSSLRSEDTAVYYCLASSYTNDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 6 aa, synthetic construct
QDVGTA

SEQ ID NO: 29 - skipped

SEQ ID NO: 30 - protein, 9 aa, synthetic construct
QQYANLPYT

SEQ ID NO: 31 - protein, 11 aa, synthetic construct
RASQDVGTAVA

SEQ ID NO: 32 - protein, 7 aa, synthetic construct
WASTRHT

SEQ ID NO: 33 - protein, 107 aa, synthetic construct; identical to SEQ ID NO: 16

SEQ ID NO: 34 - protein, 214 aa, synthetic construct; identical to SEQ ID NO: 17

SEQ ID NO: 35 - protein, 8 aa, synthetic construct
GFTFSGYT

SEQ ID NO: 36 - protein, 8 aa, synthetic construct
ISSAGSYI

SEQ ID NO: 37 - protein, 12 aa, synthetic construct
LASYITTSYFEH

SEQ ID NO: 38 - protein, 6 aa, synthetic construct
SSAGSY

SEQ ID NO: 39 - protein, 10 aa, synthetic construct
SYITTSYFEH

SEQ ID NO: 40 - protein, 5 aa, synthetic construct
GYTMS

SEQ ID NO: 41 - protein, 17 aa, synthetic construct
TISSAGSYIYYLDSVKG

SEQ ID NO: 42 - protein, 119 aa, synthetic construct
EVQLVESGGGLVKPGGSLKLSCAASGFTFSGYTMSWFRQAPEKRLEWVATISSAGSYIYYLDSVKGRFTISRDNSKNTLYLQMSSLRSEDTAVYYCLASYITTSYFEHWGQGTLVTVSS

SEQ ID NO: 43 - protein, 449 aa, synthetic construct
EVQLVESGGGLVKPGGSLKLSCAASGFTFSGYTMSWFRQAPEKRLEWVATISSAGSYIYYLDSVKGRFTISRDNSKNTLYLQMSSLRSEDTAVYYCLASYITTSYFEHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 6 aa, synthetic construct
QDVGTA

SEQ ID NO: 45 - skipped

SEQ ID NO: 46 - protein, 9 aa, synthetic construct
QQYSNLPYT

SEQ ID NO: 47 - protein, 11 aa, synthetic construct
RASQDVGTAVA

SEQ ID NO: 48 - protein, 7 aa, synthetic construct
WASTRHT

SEQ ID NO: 49 - protein, 107 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYSNLPYTFGGGTKLEIK

SEQ ID NO: 50 - protein, 214 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYSNLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 225 aa, synthetic construct
EVQLVESGGGLVKPGGSLKLSCAASGFTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 52 - protein, 214 aa, synthetic construct
DIVMTQSPSSLSASVGDRVTITCRASQDVGTAVAWYQQKPGQSPKLLIYWASTRHTGVPDRFSGSGSGTDFTLTISGLQSEDEADYFCQQYSNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 225 aa, synthetic construct
EVQLVESGGGLVKPGGSLKLSCAASGLTFSGYAMSWFRQAPEKRLEWVATISSGGSYTYYLDSVKGRFTISRDNSKNTLYLQMSSLRSEDTAVYYCLASLITTDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 54 - protein, 214 aa, synthetic construct
DIVMTQSPSSLSASVGDRVTITCRASQDVGTAVAWYQQKPGQSPKLLIYWASTRYTGVPDRFSGSGSGTDFTLTISGLQSEDEADYFCQQYSNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 225 aa, synthetic construct
EVQLVESGGGLVKPGGSLKLSCAASGFTFSDYAMSWFRQAPEKRLEWVATISSTGSYKYYLDSVKGRFTISRDNSKNTLYLQMSSLRSEDTAVYYCLASVITNDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 56 - protein, 214 aa, synthetic construct; identical to SEQ ID NO: 17

SEQ ID NO: 57 - protein, 225 aa, synthetic construct
EVQLVESGGGLVKPGGSLKLSCAASGFTFSDYAMSWFRQAPEKRLEWVATISSTGSYSYYLDSVKGRFTISRDNSKNTLYLQMSSLRSEDTAVYYCLASSYTNDYFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 58 - protein, 214 aa, synthetic construct; identical to SEQ ID NO: 17

SEQ ID NO: 59 - protein, 225 aa, synthetic construct
EVQLVESGGGLVKPGGSLKLSCAASGFTFSGYTMSWFRQAPEKRLEWVATISSAGSYIYYLDSVKGRFTISRDNSKNTLYLQMSSLRSEDTAVYYCLASYITTSYFEHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKT

SEQ ID NO: 60 - protein, 214 aa, synthetic construct; identical to SEQ ID NO: 50

(19) United States
(12) Patent Application Publication
(10) Pub. No.: US 2026/0286011 A1
(43) Pub. Date: Sep. 24, 2026

(54) ANTIBODIES OR ANTIGEN-BINDING FRAGMENTS THEREOF CAPABLE OF BINDING TO FREE DRUG AND USES THEREOF

(71) Applicant: Generate Biomedicines, Inc., Somerville, MA (US)

(72) Inventors: Melissa Leith Conerly, Issaquah, WA (US); Heather Van Epps, Arlington, MA (US); Erin C. Yang, Cambridge, MA (US); Benjamin Manning, Watertown, MA (US); Peter Bloomingdale, Saint Augustine, FL (US); Jiayu Chen, Medford, MA (US); Hideki Shishido, Belmont, MA (US); Giovanni Tosi, Brookline, MA (US); Rounak Nassirpour, Tewksbury, MA (US); Monica Menzenski, Cambridge, MA (US); Thomas William Linsky, Somerville, MA (US); Adambarage Dinesh De Alwis, New York, NY (US); Kapil Mayawala, Secaucus, NJ (US)

(21) Appl. No.: 19/420,636

(22) Filed: Dec. 15, 2025

Related U.S. Application Data
(60) Provisional application No. 63/735,826, filed on Dec. 18, 2024.

(51) Int. Cl. C07K 16/44; A61K 39/00; A61P 39/00

(52) U.S. Cl. CPC C07K 16/44; A61P 39/00; A61K 2039/505; C07K 2317/33; C07K 2317/565; C07K 2317/76; C07K 2317/92

(57) ABSTRACT

The present disclosure provides antibodies or antigen-binding fragments thereof that are capable of binding to free drug of antibody drug conjugates (ADCs), and related technologies. The present disclosure also provides methods of treating or preventing off-target effects of ADCs in a subject that involve administering a therapeutically effective amount of a pharmaceutical composition comprising an antibody or antigen-binding fragment thereof provided herein to a subject.

### CROSS-REFERENCE TO RELATED APPLICATIONS

This application claims the benefit of U.S. Provisional Application 63/735,826, filed Dec. 18, 2024, the entirety of which is incorporated herein by reference.

### SEQUENCE LISTING

The present specification makes reference to a Sequence Listing (submitted electronically as an .xml file named 2017408-0047_SL.xml on Jun. 10, 2026). The .xml file was generated on Mar. 10, 2026, and is 55,693 bytes in size. The entire contents of the Sequence Listing are herein incorporated by reference.

### BACKGROUND

Antibody-drug conjugates (ADCs) can provide an effective means of delivering a drug to a targeted site in a tissue or organism. Nonetheless, ADCs can result in adverse effects associated with toxicity of “free” drugs (e.g., unbound to a carrier, such as an antibody or antigen-binding fragment thereof) in off-target tissues. Therefore, a need exists for development of new therapeutic modalities capable of reducing off-target toxicity of ADCs by binding to free drugs in the treatment of diseases, disorders, and conditions.

### SUMMARY

The present disclosure provides antibodies or antigen-binding fragments thereof capable of binding to a free drug (e.g., monomethyl auristatin E (MMAE)) and reducing off-target toxicity of a free drug of antibody drug conjugates (ADCs). ADCs are designed to maximize a therapeutic window by delivering chemotherapeutic drugs specifically to tumors with reduced impact on normal tissues (e.g., off-target tissues). Use of ADCs can be limited by off-target toxicities (e.g., one or both of peripheral neuropathy and neutropenia) mediated by release of a free drug (e.g., MMAE), resulting in a narrow therapeutic window. In some embodiments, an antibody or antigen-binding fragment thereof described herein exhibits one or more of the following characteristics: (a) specific binding to a free drug of an ADC; (b) preventing off-target toxicity of a free drug of an ADC; and (c) no substantial inhibition of ADC activity. In some embodiments, an antibody or antigen-binding fragment thereof reduces off-target toxicities (e.g., one or both of peripheral neuropathy and neutropenia) of a free drug of an ADC in a subject.

In some embodiments, an antibody or antigen-binding fragment thereof capable of binding to a free drug of an antibody drug conjugate (ADC), comprises: (a) a heavy chain variable region (VH) comprising a VHCDR1 amino acid sequence of SEQ ID NO: 18; a VHCDR2 amino acid sequence of SEQ ID NO: 19; and a VHCDR3 amino acid sequence of SEQ ID NO: 20; and a light chain variable region (VL) comprising a VLCDR1 amino acid sequence of SEQ ID NO: 28, a VLCDR2 amino acid sequence of SEQ ID NO: 29, and a VLCDR3 amino acid sequence of SEQ ID NO: 30; (b) a VH comprising a VHCDR1 amino acid sequence of SEQ ID NO: 21; a VHCDR2 amino acid sequence of SEQ ID NO: 22; and a VHCDR3 amino acid sequence of SEQ ID NO: 23; and a VL comprising a VLCDR1 amino acid sequence of SEQ ID NO: 31, a VLCDR2 amino acid sequence of SEQ ID NO: 32, and a VLCDR3 amino acid sequence of SEQ ID NO: 30; (c) a VH comprising a VHCDR1 amino acid sequence of SEQ ID NO: 24; a VHCDR2 amino acid sequence of SEQ ID NO: 25; and a VHCDR3 amino acid sequence of SEQ ID NO: 23; and a VL comprising a VLCDR1 amino acid sequence of SEQ ID NO: 31, a VLCDR2 amino acid sequence of SEQ ID NO: 32, and a VLCDR3 amino acid sequence of SEQ ID NO: 30; (d) a VH comprising a VHCDR1 amino acid sequence of SEQ ID NO: 1; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 11, a VLCDR2 amino acid sequence of SEQ ID NO: 12, and a VLCDR3 amino acid sequence of SEQ ID NO: 13; (e) a VH comprising a VHCDR1 amino acid sequence of SEQ ID NO: 4; a VHCDR2 amino acid sequence of SEQ ID NO: 5; and a VHCDR3 amino acid sequence of SEQ ID NO: 6; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 13; (f) a VH comprising a VHCDR1 amino acid sequence of SEQ ID NO: 7; a VHCDR2 amino acid sequence of SEQ ID NO: 8; and a VHCDR3 amino acid sequence of SEQ ID NO: 6; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 13; (g) a VH comprising a VHCDR1 amino acid sequence of SEQ ID NO: 35; a VHCDR2 amino acid sequence of SEQ ID NO: 36; and a VHCDR3 amino acid sequence of SEQ ID NO: 37; and a VL comprising a VLCDR1 amino acid sequence of SEQ ID NO: 44, a VLCDR2 amino acid sequence of SEQ ID NO: 45, and a VLCDR3 amino acid sequence of SEQ ID NO: 46; (h) a VH comprising a VHCDR1 amino acid sequence of SEQ ID NO: 35; a VHCDR2 amino acid sequence of SEQ ID NO: 38; and a VHCDR3 amino acid sequence of SEQ ID NO: 39; and a VL comprising a VLCDR1 amino acid sequence of SEQ ID NO: 47, a VLCDR2 amino acid sequence of SEQ ID NO: 48, and a VLCDR3 amino acid sequence of SEQ ID NO: 46; or (i) a VH comprising a VHCDR1 amino acid sequence of SEQ ID NO: 40; a VHCDR2 amino acid sequence of SEQ ID NO: 41; and a VHCDR3 amino acid sequence of SEQ ID NO: 39; and a VL comprising a VLCDR1 amino acid sequence of SEQ ID NO: 47, a VLCDR2 amino acid sequence of SEQ ID NO: 48, and a VLCDR3 amino acid sequence of SEQ ID NO: 46.

In some embodiments, an antibody or antigen-binding fragment thereof capable of binding to a free drug of an antibody drug conjugate (ADC), comprises: (a) a VH comprising an amino acid sequence having at least about 85% sequence identity to an amino acid sequence of any one of SEQ ID NOs: 9, 26, or 42; and (b) a VL comprising an amino acid sequence having at least about 85% sequence identity to an amino acid sequence of any one of SEQ ID NOs: 16, 33, or 49.

In some embodiments, an antibody or antigen-binding fragment thereof comprises: (a) a VH comprising an amino acid sequence of any one of SEQ ID NOs: 9, 26, or 42; and (b) a VL comprising an amino acid sequence of any one of SEQ ID NOs: 16, 33, or 49. In some embodiments, an antibody or antigen-binding fragment there of comprises: (a) a VH comprising an amino acid sequence of SEQ ID NO: 9 and a VL comprising an amino acid sequence of SEQ ID NO: 16; (b) a VH comprising an amino acid sequence of SEQ ID NO: 26 and a VL comprising an amino acid sequence of SEQ ID NO: 33; or (c) a VH comprising am amino acid sequence of SEQ ID NO: 42 and a VL comprising an amino acid sequence of SEQ ID NO: 49. In some embodiments, an antibody or antigen-binding fragment thereof is: (i) a humanized antibody or antigen-binding fragment thereof, and/or (ii) a monospecific antibody or antigen-binding fragment thereof, or a bispecific antibody or antigen-binding fragment thereof.

In some embodiments, an antibody or antigen-binding fragment thereof is: (i) a humanized antibody or antigen-binding fragment thereof, and/or (ii) a monospecific antibody or antigen-binding fragment thereof, or a bispecific antibody or antigen-binding fragment thereof. In some embodiments, an antibody or antigen-binding fragment thereof comprises a Fab, F(ab′)₂, Fv, or a single chain Fv fragment (scFv). In some embodiments, an antibody or antigen-binding fragment thereof comprises: (i) a heavy chain constant region selected from IgG1, IgG2, IgG3, and IgG4, and/or (ii) a light chain constant region chosen from kappa or lambda.

In some embodiments, an antibody or antigen-binding fragment there of comprises: (a) a heavy chain comprising an amino acid sequence with at least about 80% sequence identity to an amino acid sequence of any one of SEQ ID NOs: 10, 27, or 43; and (b) a light chain comprising an amino acid sequence with at least about 80% sequence identity to an amino acid sequence of any one of SEQ ID NOs: 17, 34, or 50. In some embodiments, an antibody or antigen-binding fragment thereof comprises: (a) a heavy chain comprising an amino acid sequence of any one of SEQ ID NOs: 10, 27, or 43; and (b) a light chain comprising an amino acid sequence of any one of SEQ ID NOs: 17, 34, or 50. In some embodiments, an antibody or antigen-binding fragment thereof comprises: (a) a heavy chain comprising the amino acid sequence of SEQ ID NO: 10 and a light chain comprising the amino acid sequence of SEQ ID NO: 17; (b) a heavy chain comprising the amino acid sequence of SEQ ID NO: 27 and a light chain comprising the amino acid sequence of SEQ ID NO: 34; or (c) a heavy chain comprising the amino acid sequence of SEQ ID NO: 43 and a light chain comprising the amino acid sequence of SEQ ID NO: 50.

In some embodiments, a drug is an auristatin. In some embodiments, an auristatin is monomethyl auristatin E (MMAE), monomethyl auristatin F (MMAF), or monomethyl auristatin D (MMAD).

In some embodiments, a pharmaceutical composition disclosed herein comprises an antibody or antigen-binding fragment thereof provided herein and a pharmaceutically acceptable carrier. In some embodiments, a polynucleotide disclosed herein encodes an antibody or antigen-binding fragment thereof provided herein. In some embodiments, an expression vector disclosed herein comprises a polynucleotide provided herein. In some embodiments, a host cell disclosed herein comprises an expression vector provided herein. In some embodiments, a method of making an antibody or antigen-binding fragment thereof disclosed herein comprises culturing a host cell described herein under conditions suitable for gene expression.

In some embodiments, a method of treating or preventing off-target effects of a free drug in a subject comprises administering a therapeutically effect amount of a pharmaceutical composition disclosed herein to a subject. In some embodiments, a free drug was conjugated to an ADC. In some embodiments, a method of treating a subject having a disease, disorder, or condition comprising administering a therapeutically effective amount of a pharmaceutical composition disclosed herein to a subject.

In some embodiments, a subject has or is at risk of developing cancer. In some embodiments, a subject has a solid tumor or a hematological cancer. In some embodiments, a solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, a thyroid cancer, a prostate cancer, a bladder cancer, a brain cancer, a retinoblastoma cancer, an esophageal cancer, a gastric cancer, a stomach cancer, a testicular cancer, a head and neck cancer, a metastatic cancer, a neuroblastoma cancer, a pancreatic cancer, or a small intestine cancer. In some embodiments, a hematological cancer comprises or is a leukemia or lymphoma. In some embodiments, a bladder cancer is a muscle-invasive bladder cancer (MIBC).

### BRIEF DESCRIPTION OF THE DRAWING

The Figures described below, which together make up the Drawing, are for illustration purposes only, not for limitation.

FIG. 1 shows EC50 values from the DELFIA binding assay with an 8-point dilution series and duplicate wells for reference antibody and exemplary neutralizing antibody variants. EC50 values for reference antibody on each assay plate are shown on the left-hand side, and EC50 values for each exemplary neutralizing antibody variant are shown on the right-hand side. The broken line indicates mean EC50 value for the reference antibody. MMAF-PEG2-Biotin is custom made from Anthem Biosciences and was used as a coating antigen.

FIG. 2 shows dose response binding of Exemplary Payload Neutralizing Antibodies 4, 5, and 6, and reference antibody characterized by a DELFIA binding assay. Curves represent each antibody. Binding of exemplary payload neutralizing antibodies to MMAF-biotin-streptavidin increased in a dose-dependent manner.

FIGS. 3A-3C show specific neutralization of monomethyl auristatin E (MMAE) cytotoxicity without impairment of antibody drug conjugated (ADC) activity. Karpas 299 cells were incubated with MMAE (0.5 nM) and neutralizing antibody variants or reference antibody (0.01 nM-28 nM) over a 72-hour incubation period (FIG. 3A). Exemplary payload neutralizing antibodies had lower IC50 values relative to Reference IgG (bottom dashed line, inverted triangle) and Reference Fab (top dashed line, triangle), indicating that free MMAE was rendered inactive for the duration of the assay (FIG. 3B). Karpas 299 cells were incubated with Brentuximab Vedotin (BV) (0.08 nM), an anti-CD30 ADC with MMAE payload, and exemplary payload neutralizing antibody variants or reference antibody (0.01 nM-28 nM) over a 72-hour incubation period. Cytotoxicity of BV was not interfered with by addition of exemplary payload neutralizing antibody variants. (FIG. 3C).

FIGS. 4A-4C show a cytotoxicity assay illustrating that Exemplary Payload Neutralizing Antibody 2 exhibits strong MMAE neutralization relative to reference antibodies over a 120-hour incubation period. Karpas 299 cells were incubated with MMAE (0.5 nM), an affinity matured reference (Reference Antibody 2), and Exemplary Payload Neutralizing Antibodies 1, 2, or 3, or reference antibody (Reference Antibody 1; 0.01 nM-28 nM; FIG. 4A). Karpas 299 cells were incubated with Brentuximab Vedotin (0.08 nM), an affinity matured reference (Reference Antibody 2), and Exemplary Payload Neutralizing Antibodies 1, 2, or 3, or reference antibody (Reference Antibody 1) (0.01 nM-28 nM) (FIG. 4B). FIG. 4C shows a cytotoxicity assay illustrating that Exemplary Payload Neutralizing Antibody 2 blocked MMAE but not ADC cytotoxicity. Viability of HPAFII and HT-1376 cells incubated with 2 nM MMAE or 100 nM EV was assessed in the presence of increasing concentrations of Exemplary Payload Neutralizing Antibody 2 lacking LALAPA Fc mutations.

FIG. 5 shows that binding of Exemplary Payload Neutralizing Antibodies 1, 2, and 3 to immobilized MMAF (MMAF-biotin-streptavidin) decreased with increasing concentrations of free MMAE with IC50 values of 5.0, 3.2, and 4.4 nM, respectively, whereas Brentuximab Vedotin (ADC) did not compete for binding.

FIG. 6 shows that binding of exemplary payload neutralizing antibody variants to a payload (MMAF-PEG2-Biotin) was measured using an AlphaLISA homogeneous binding assay with an 8-point dilution series. Curves represent each exemplary payload neutralizing antibody variant. Binding of exemplary payload neutralizing antibody variants to MMAF-biotin-streptavidin increased in a dose-dependent manner, with a hook effect observed at higher concentrations. Results are shown as mean of n=3 technical replicates, fitted using a dose-response stimulation curve with a bell-shaped model in GraphPad Prism 10 software.

FIG. 7 shows pharmacokinetics of five exemplary payload neutralizing antibodies in mice. Female SCID mice (n=4) were randomized based on body weight and received a single intravenous dose of 10 mg/kg of an exemplary payload neutralizing antibody. Plasma-EDTA samples were collected at multiple time points: 1 hour, 4 hours, 24 hours, 3 days, and 7 days post-injection.

FIG. 8 shows pharmacokinetics of Exemplary Payload Neutralizing Antibody 1 in mice. Female SCID mice (7-8 weeks old) were randomized based on body weight and injected intravenously with either BV alone or a combination of BV and payload neutralizing antibodies. Plasma-EDTA samples were collected at 24 hours, 7 days, and 28 days.

FIGS. 9A-9B show pharmacokinetics/pharmacodynamics of Exemplary Payload Neutralizing Antibody 1 in mice. Female SCID mice (7-8 weeks old) were randomized based on body weight and injected intravenously with either BV alone or a combination of BV and Exemplary Payload Neutralizing Antibody 1. Plasma-EDTA samples were collected at 15 minutes, 8 hours, 24 hours, 48 hours, 72 hours, and 7 days. FIG. 9A shows levels of free MMAE in circulation. FIG. 9B shows percent reduction in Free MMAE Exposure by Payload Neutralizing Antibody 1 relative to ADC alone.

FIG. 10 shows the effect of Exemplary Payload Neutralizing Antibody 1-mediated reduction of free MMAE on ADC activity using a patient-derived xenograft (PDX) murine model of bladder cancer (ST458). Anti-Nectin4 ADC, Enfortumab vedotin, was administered alone, or in combination with Exemplary Payload Neutralizing Antibody 1 in a patient-derived xenograft model of bladder cancer (ST458). Female athymic nude (Crl:NU(NCr)-Foxn1^{nu}) were implanted with a PDX model ST458 subcutaneously in one flank following IACUC protocols. Tumor volumes were recorded every 3-4 days, and animals were removed from the study when tumors reached 2500 mm³.

FIG. 11 shows an equilibrium binding curve of Exemplary Payload Neutralizing Antibody 2 lacking LALPA Fc mutations binding to MMAE. Curve 1 (dark) represents the KD-controlled curve while curve 2 (light) represents the concentration-controlled curve, each fit to a 1:1 reversible bimolecular interaction using MMAE as the concentration reference. Each curve is an independent experiment with technical replicates.

FIG. 12 shows normalized responses of Exemplary Payload Neutralizing Antibody 2, isotype-hlgG1 and isotype-hIgG1-LALAPA binding to a panel of recombinant human Fc gamma receptors at 5000 nM, measured by SPR. Antibody binding responses were normalized to human Fc gamma receptor capture levels and reported as normalized binding responses with the standard deviation of one experiment with technical replicates. Cutoff of ≤0.06 (equivalent to ≤5 RU at 75 RU capture) was considered non-binding interaction.

FIG. 13 shows responses of human Clq binding to Exemplary Payload Neutralizing Antibody 2, isotype-hIgG1 and isotype-hIgG1-LALAPA measured by BLI. The dotted line is the cutoff positioned at 0.1 nm to indicate a non-binding interaction (≤0.1 nm) from a binding interaction (>0.1 nm).

FIGS. 14A-B show the effect of Exemplary Payload Neutralizing Antibody 1-mediated reduction of free MMAE on ADC activity using a patient-derived xenograft (PDX) murine model of bladder cancer (ST458). Anti-Nectin4 ADC, Enfortumab vedotin, (EV) was administered alone, or in combination with Exemplary Payload Neutralizing Antibody 1 in a patient-derived xenograft model of bladder cancer (ST458). Female athymic nude (Crl:NU(NCr)-Foxn1^{nu}) were implanted with a PDX model ST458 subcutaneously in one flank following IACUC protocols. Tumor volumes were recorded every 3-4 days, and animals were removed from the study when tumors reached 2500 mm³. FIG. 14A shows mean tumor volume over time for mice bearing ST458 PDX tumors. Data points are means +/−SEM (N=5-12 mice per group—see Table 7). Arrows indicate timing of intravenous dosing. The curves were truncated on the day the first mouse in the group reached maximum tumor volume. FIG. 14B shows pharmacokinetics of free plasma MMAE in mice treated with 3 mg/kg EV alone or in combination with 0.3 mg/kg or 3 mg/kg of Exemplary Payload Neutralizing Antibody 1 (left panel); the levels of free MMAE in the group treated with 30 mg/kg Exemplary Payload Neutralizing Antibody 1 were below limit of quantification (LOQ) at every time point measured. The dashed horizontal line indicates the LOQ (10 pg/mL). The right panel shows free plasma MMAE exposures (AUC) across two different time periods, from hour 0 to 168 (“AUC0-168”) or from hour 0 to 368 (“AUC0-last”), normalized to EV alone group.

FIG. 15 shows pharmacokinetics of Exemplary Antibody 2 in tumor-bearing mice. Mice were co-administered 3 mg/kg Enfortumab vedotin (EV) and Exemplary Antibody 2 intravenously once every 7 days for 3 doses. Exemplary Antibody 2 was evaluated in mouse plasma across five dose levels (0.1, 0.3, 1, 3, and 10 mg/kg).

FIGS. 16A-B show the effect of Exemplary Antibody 2-mediated reduction of free MMAE on Enfortumab vedotin (EV) antitumor activity. FIG. 16A shows mean tumor volume over time for mice bearing ST458 PDX tumors. Data points are means+/−SEM (N=9-15 mice per group). Arrows indicate intravenous dosing. The curves were truncated at the timepoint when the first mouse in the group reached maximum tumor volume (2500 mm³). FIG. 16B shows pharmacokinetics of free plasma MMAE (left panel) and dose-dependent reduction in free plasma MMAE caused by Exemplary Antibody 2 (right panel) in mice for six different treatment arms: 3 mg/kg EV in combination with 10 mg/kg isotype control or 0.1, 0.3, 1, 3, and 10 mg/kg of Exemplary Antibody 2. EV, Exemplary Antibody 2, and isotype controls were administered intravenously weekly for 3 weeks. The dashed horizontal line marks the limit of quantification (LOQ) for free MMAE concentrations (10 pg/mL). Concentrations below LOQ were set to the LOQ of 10 pg/mL. Dose-dependent reduction in free plasma MMAE exposure caused by Exemplary Antibody 2 is presented as the ratio of the area under the concentration-time curve (AUC) for each treated arm relative to 3 mg/kg EV combined with 10 mg/kg isotype control, across three intervals: day 0-7 (AUC_{0-1week}), day 0-14 (AUC_{0-2-week}), and day 0-28 (AUC₀₋ₗₐₛₜ).

### Definitions

In order for various aspects described herein to be more readily understood, certain terms are first defined below. Additional definitions for the following terms and other terms are set forth throughout the specification. The publications and other reference materials referenced herein describe the background of various aspects described herein and provide additional detail regarding its practice are hereby incorporated by reference.

The articles “a” and “an” are used herein to refer to one or to more than one (i.e., to at least one) of the grammatical object of the article. By way of example, “an agent” means one agent or more than one agent.

About: As used herein, the term “about,” as applied to one or more values of interest, refers to a value that is similar to a stated reference value. In some embodiments, the term “about” refers to a range of values that fall within about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, or less in either direction (greater than or less than) of the stated reference value unless otherwise stated or otherwise evident from the context (except where such number would exceed about 100% of a possible value).

Antibody: As used herein, the term “antibody” refers to a polypeptide that includes canonical immunoglobulin sequence elements sufficient to confer specific binding to a particular target antigen. In some embodiments, an antibody refers to an anti-Spike protein antibody or antigen-binding fragment thereof. Intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch,” connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., about 3-, about 4-, or about 5-stranded sheets) packed against each other in a compressed antiparallel beta barrel.

In some embodiments, an antibody or antigen-binding fragment thereof is or comprises a polypeptide whose amino acid sequence includes structural elements recognized by those skilled in the art as an immunoglobulin variable domain. In some embodiments, an antibody or antigen-binding fragment thereof is a polypeptide protein having a binding domain that is homologous or largely homologous to an immunoglobulin-binding domain. When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with various aspects described herein include glycosylated Fc domains, such as Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen) or produced by recombinant engineering, chemical synthesis, or other artificial system or methodology. In some embodiments, an antibody is polyclonal. In some embodiments, an antibody is monoclonal. In some embodiments, an antibody has constant region sequences characteristic of mouse, rabbit, primate, or human antibodies. In some embodiments, antibody sequence elements are humanized, primatized, or chimeric

Moreover, the term “antibody,” as used herein, can refer in appropriate embodiments (unless otherwise stated or clear from context) to any of the art-known or developed constructs or formats for utilizing antibody structural and functional features in alternative presentation. For example, in some embodiments, an antibody provided herein is utilized in accordance with a format selected from, but not limited to, intact IgA, IgG, IgE or IgM antibodies; bispecific or multi-specific antibodies (e.g., Zybodies®, etc.); and/or antibody fragments (preferably antibody fragments that exhibit desired antigen-binding activity). An antibody described herein can be an immunoglobulin, heavy chain antibody, light chain antibody, LRR-based antibody, or other protein scaffold with antibody-like properties, as well as any other known immunological binding moiety, e.g., a Fab, Fab′, Fab′2, Fab2, Fab3, F(ab′)2, Fd, Fv, Feb, scFv, SMIP, antibody, diabody, triabody, tetrabody, minibody, maxibody, tandab, DVD, BiTe, TandAb, or any combination thereof. The subunit structures and three-dimensional configurations of different classes of antibodies are known. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Antibody drug conjugate: As used herein, the term “antibody drug conjugate” or “ADC” refers to a reagent made up of an antibody or antigen-binding fragment thereof chemically linked to a drug. In some embodiments, an antibody or antigen-binding fragment thereof binds to specific proteins or receptors found on certain types of cells (e.g., cancer cells). An associated drug enters these cells and kills them without harming other cells. In some embodiments, a free drug (e.g., unbound to a carrier, such as an antibody or antigen-binding fragment thereof) harms other cells creating adverse effects. A free drug is also referred to herein as a payload. In some embodiments, ADCs are used to treat a disease, disorder, or condition (e.g., cancer).

Antibody heavy chain: As used herein, the term “antibody heavy chain” refers to the larger of the two types of polypeptide chains present in all antibodies in their naturally occurring conformations.

Antibody light chain: As used herein, the term “antibody light chain” refers to the smaller of the two types of polypeptide chains present in all antibodies in their naturally occurring conformations.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen” as that term is used herein. Furthermore, one skilled in the art will understand that an antigen need not be encoded solely by a full-length nucleotide sequence of a gene. It is readily apparent that the various aspects of innovations described herein includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell, or a biological fluid.

Antigen-binding fragment: As used herein, the term “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2, diabodies, linear antibodies, single-chain antibody molecules (e.g., scFv, VHH, camelid, or VH or VL domains only), or multispecific antibodies formed from antibody fragments. In some embodiments, antigen-binding fragments of antibodies described herein are scFvs. In some embodiments, antigen-binding fragments of antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen. An antigen-binding fragment may be produced by any means. For example, in some embodiments, an antigen-binding fragment is enzymatically or chemically produced by fragmentation of an intact antibody. Alternatively, in some embodiments, an antigen-binding fragment is recombinantly produced. In some embodiments, an antigen-binding fragment is wholly or partially synthetically produced. In some embodiments, an antigen-binding fragment has a length of at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, or at least about 200 amino acids or more.

Associated: Two events or entities are “associated” with one another, as that term is used herein, if the presence, level, degree, type and/or form of one is correlated with that of the other. For example, a particular entity (e.g., polypeptide, genetic signature, metabolite, or microbe) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities physically associated with one another are covalently linked to one another. In some embodiments, two or more entities physically associated with one another are not covalently linked to one another but are non-covalently associated, for example, by means of hydrogen bonds, van der Waals interaction, hydrophobic interactions, magnetism, and combinations thereof.

Binding: As used herein, the term “binding” refers to a non-covalent association between or among two or more entities. “Direct” binding involves physical contact between entities or moieties. Indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

CDR: As used herein, “CDR” refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. In general, there are three CDRs in each heavy chain variable region (HCDR1, HCDR2, HCDR3) and three CDRs in each light chain variable region (LCDR1, LCDR2, LCDR3). The exact definitional CDR boundaries and lengths are subject to different classification and numbering systems. Certain systems have been established for defining CDR boundaries (e.g., Kabat, IMGT, Chothia, or a combination thereof). CDRs may therefore be referred to by Kabat, Chothia, IMGT, or any other known boundary definitions. Despite differing boundaries, each of these systems has some degree of overlap in what constitutes the “hypervariable regions” within the variable sequences. CDR definitions according to these systems may therefore differ in length and boundary areas with respect to the adjacent framework region (see, e.g., Kabat et al., 1992, “Sequences of Proteins of Immunological Interest,” 5th Edition, U.S. Department of Health and Human Services; Chothia et al., 1987, J. Mol. Biol. 196:901; and MacCallum et al., 1996, J. Mol. Biol. 262:732, each of which is incorporated by reference in its entirety). Those skilled in the art appreciate the differences between and among these systems and are capable of understanding CDR boundaries to the extent required to understand and to practice the claims and various aspects of innovations described herein.

Chimeric antibody: As used herein, the term “chimeric antibody” refers to an antibody that has framework residues from one species, such as human, and complementarity-determining regions (CDRs, which generally confer antigen binding) from another species, such as a murine antibody that specifically binds a payload. In a chimeric antibody, some portions of heavy and/or light chains may be identical or homologous to sequences from a particular species while other portions may be identical or homologous to sequences from a different species. Chimeric antibodies generally exhibit decreased immunogenicity and increased stability. Techniques for cloning murine immunoglobulin variable domains known in the art (see Orlandi et al., 1989, PNAS. 86:3833, and Leung et al., 1994, Hybridoma. 13:469, each of which is hereby incorporated by reference in its entirety). As an example of a chimeric antibody, polynucleotides encoding variable domains of a light chain or heavy chain of an antibody derived from an animal (e.g., mouse, rat, or chicken) other than human can be linked to polynucleotides encoding t constant domains of a light chain or heavy chain derived from a human antibody to produce a polynucleotide (such as DNA) encoding a chimeric antibody.

Composition: As used herein, the term “composition” is known by those skilled in the art, who will appreciate that the term “composition” may be used to refer to a discrete physical entity that comprises one or more specified components. In general, unless otherwise specified, a composition may be of any form—e.g., gas, gel, liquid, or solid.

Comprising: As used herein, the term “comprising” in reference to a composition or method described herein refers to one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method described herein, known or disclosed equivalents of any named essential element or step may be substituted for that element or step.

Fragment: As used herein, the term “fragment” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, an antigen-binding fragment comprises or consists of at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 275, at least about 300, at least about 325, at least about 350, at least about 375, at least about 400, at least about 425, at least about 450, at least about 475, at least about 500, or more monomeric units (e.g., amino acids) as found in a whole antibody. In some embodiments, an antigen-binding fragment comprises or consists of at least about 5%, at least about 10%, at least about 15%, at least about 20%, at least about 25%, at least about 30%, at least about 25%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more of the monomeric units (e.g., residues) found in a whole antibody. In some embodiments, a nucleotide fragment comprises or consists of at least about 5%, at least about 10%, at least about 15%, at least about 20%, at least about 25%, at least about 30%, at least about 25%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more of the monomeric units (e.g., residues) found in the whole nucleotide.

Identity: As used herein, the term “identity” refers to the subunit sequence identity between two polymeric molecules, particularly between two amino acid molecules, such as between two polypeptide molecules. When two amino acid sequences have the same residues at the same positions, e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half of the positions (e.g., five positions in a polymer of 10 amino acids in length) in two sequences are identical, the two sequences are 50% identical; if 90% of the positions (e.g., nine positions in a polymer of 10 amino acids in length) are identical, the two amino acids sequences are 90% identical.

Off-target: As used herein, the term “off-target”, in reference to toxicity, refers to toxicity that accompanies administration of many chemotherapeutic agents (e.g., ADCs). While the intended purpose of administration of a chemotherapeutic agent is to reduce or inhibit growth of tumors or any accompanying metastases, the growth, function, and/or physiology of normal cells are often adversely affected during treatment of cancer. A reduction of off-target toxicity is intended to reduce any adverse effects on non-tumor or non-metastatic cells.

Pharmaceutically acceptable: As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into monomeric “nucleotides.” Monomeric nucleotides can be hydrolyzed into nucleosides. As used herein polynucleotides include, but are not limited to, all nucleic acid sequences which are obtained by any means available in the art, including, without limitation, recombinant means, e.g., cloning of nucleic acid sequences from a recombinant library or a cell genome, using ordinary cloning technology and PCR™, and the like, and by synthetic means.

Prevent: As used herein, the term “prevent” when used in connection with the occurrence of disease, disorder, or condition, refers to reducing the risk of developing a disease, disorder, or condition (e.g., related to off-target effects of a free drug of an ADC) and/or to delaying onset of one or more characteristics or symptoms of a disease, disorder, or condition (e.g., related to off-target effects of a free drug of an ADC). In some embodiments, preventing refers to neutralizing a free drug (e.g., MMAE) of an ADC. Prevention may be considered complete when onset of a disease, disorder, or condition has been delayed for a predefined period of time.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder, or condition, e.g., a disease, disorder, or condition that can be treated as provided herein. In some embodiments, a subject is susceptible to a disease, disorder, or condition. In some embodiments, a susceptible subject is predisposed to and/or shows an increased risk (as compared to the average risk observed in a reference subject or population) of developing a disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target: As used herein, the term “target” refers to an agent, cell, tissue, organ, or site within the body that is the subject of provided methods, systems, compounds, and/or compositions, for example, a cell, tissue, organ or site within a body that is in need of treatment or is an off-target, for example, a cell, tissue, organ, or site within a body targeted by a free drug (e.g., MMAE) of an ADC.

Treat: As used herein, the terms “treat,” “treatment,” or “treating” refer to partial or complete alleviation, amelioration, delay of onset of, inhibition, prevention, relief, and/or reduction in incidence and/or severity of one or more symptoms or features of a disease, disorder, and/or condition described herein (e.g., a cancer). In some embodiments, treatment is administered to a subject who does not exhibit signs or features of a disease, disorder, and/or condition (e.g., may be prophylactic). In some embodiments, treatment is administered to a subject who exhibits only early or mild signs or features of the disease, disorder, and/or condition, for example for the purpose of decreasing the risk of developing pathology associated with the disease, disorder, and/or condition. In some embodiments, treatment is administered to a subject who exhibits established, severe, and/or late-stage signs of the disease, disorder, or condition (e.g., a cancer). In some embodiments, treating comprises administering at least one antibody or antigen-binding fragment thereof described herein to a subject.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are antibodies or antigen-binding fragments thereof that bind specifically to a free drug (e.g., MMAE) of an antibody drug conjugate (ADC) and reduce off-target toxicity of an ADC, without reducing potency of an ADC, and methods of use thereof. In some embodiments, an antibody or antigen-binding fragment thereof described herein exhibits one or more of the following characteristics: (a) specific binding to a free drug of an ADC; (b) preventing off-target toxicity of a free drug of an ADC; and (c) no substantial inhibition of ADC activity. In some embodiments, an antibody or antigen-binding fragment thereof reduces off-target toxicities (e.g., peripheral neuropathy and neutropenia) of a free drug of an ADC in a subject. In some embodiments, an antibody or antigen-binding fragment thereof reduces off-target toxicities (e.g., one or both of peripheral neuropathy and neutropenia) of a free drug of an ADC in a subject by at least about 5%, at least about 10%, at least about 15%, at least about 20%, at least about 30%, at least about 40%, at least about 50%, or greater relative to free drug without an antibody or antigen-binding fragment thereof described herein. In some embodiments, an antibody or antigen-binding fragment thereof provided herein binds specifically to an auristatin. In some embodiments, an antibody or antigen-binding fragment thereof provided herein binds specifically to one or more of monomethyl auristatin E (MMAE), monomethyl auristatin F (MMAF), and monomethyl auristatin D (MMAD).

### Antibodies and Antigen-Binding Fragments Thereof

Provided herein, among other things, are antibodies or antigen-binding fragments thereof capable of binding to a free drug of an antibody drug conjugate (ADC). In some embodiments, an antibody or antigen-binding fragment thereof described herein can be or comprise an immunoglobulin, heavy chain antibody, light chain antibody, or other protein scaffold with antibody-like properties, as well as other immunological binding moiety, including a Fab fragment, a Fab′ fragment, a F(ab′)2 fragment, a Fv fragment, a disulfide-bonded Fv fragment, a scFv fragment, a diabody, a triabody, a tetrabody, a minibody, a maxibody, a tandab, BiTe, or any combination thereof.

In some embodiments, an antibody or antigen-binding fragment thereof described herein comprises or is a monoclonal antibody. In some embodiments, antibody or antigen-binding fragment thereof described herein comprises or is a full-length antibody, e.g., comprising an immunoglobulin Fc region. In some embodiments, an antibody or antigen-binding fragment thereof described herein comprises or is a multispecific antibody, e.g., comprising a plurality of immunoglobulin variable domain sequences, wherein a first immunoglobulin variable domain sequence of a plurality has binding specificity for a first epitope and a second immunoglobulin variable domain sequence of a plurality has binding specificity for a second epitope. In some embodiments, an antibody or antigen-binding fragment thereof described herein comprises or is a bispecific antibody molecule. In some embodiments, an antibody or antigen-binding fragment thereof described herein is or has been affinity matured.

An antibody or antigen-binding fragment thereof can include a heavy chain variable domain sequence (VH), and a light chain variable domain sequence (VL). In some embodiments, an antibody or antigen-binding fragment thereof comprises an immunoglobulin molecule of four polypeptide chains, e.g., two heavy chains and two light chains. A heavy chain can include a VH and a heavy chain constant domain. A heavy chain constant domain can include CH1, hinge, CH2, CH3, and optionally, a CH4 region. A light chain can include a VL and a light chain constant domain. A light chain constant domain can include a CL domain.

A VH and/or a VL can be further subdivided into regions of variability, termed complementarity determining regions (CDRs), interspersed with regions that are more conserved, termed framework regions (FR). Such VH and/or VL domains can each include three CDRs and four framework regions, arranged from amino-terminus to carboxyl-terminus in the following order: FR1, CDR1, FR2, CDR2, FR3, CDR3, FR4, one or more of which can be engineered as described herein. In general, there are three CDRs in each VH (HCDR1, HCDR2, and HCDR3) and three CDRs in each VL (LCDR1, LCDR2, and LCDR3). The extent of a framework region and CDRs can be defined using a number of well-known schemes (see, e.g., Kabat et al., 1991, “Sequences of Proteins of Immunological Interest,” Fifth Edition, U.S. Department of Health and Human Services; Chothia et al., 1987, J. Mol. Biol. 196:901-917; and the AbM definition used by Oxford Molecular's AbM antibody modeling software, each of which is hereby incorporated by reference in its entirety).

An antibody or antigen-binding fragment thereof described herein can be from any class of antibodies including, but not limited to, IgG, IgA, IgM, IgD, and IgE, and from any subclass (e.g., IgG1, IgG2, IgG3, and IgG4) of antibodies. An antibody or antigen-binding fragment thereof described herein can be or comprise a human, humanized, CDR-grafted, or in vitro generated antibody. An antibody or antigen-binding fragment thereof described herein can have or comprise a heavy chain constant region chosen from, e.g., IgG1, IgG2, IgG3, or IgG4. An antibody or antigen-binding fragment thereof can have or comprise a light chain chosen from, e.g., kappa or lambda.

In some embodiments, an antibody or antigen-binding fragment thereof described herein is or comprises a monoclonal antibody. Typically, monoclonal antibodies are obtained from a population of substantially homogeneous antibodies, such that individual antibodies comprising a population are substantially identical, except for possible naturally occurring mutations that may be present in minor amounts. Thus, a modifier “monoclonal” as used herein, indicates a character of an antibody as not being a mixture of discrete antibodies. In some embodiments, monoclonal antibodies directed to a particular epitope are derived from a single cell line (e.g., a B cell line).

In some embodiments, an antibody or antigen-binding fragment thereof described herein is or comprises a polyclonal antibody. In contrast to monoclonal antibodies, polyclonal antibodies are typically obtained from a population of heterogeneous antibodies, such that antibodies in a particular population include structural variation, for example, affinity for different binding sites on a particular target (e.g., a free drug of an ADC). Several methods of producing polyclonal antibodies include use of multiple subcutaneous and/or intraperitoneal injections of a relevant antigen into an animal, optionally including co-administration of one or more adjuvants.

Examples of antibodies or antigen-binding fragments thereof described herein can include: (i) a Fab fragment, a monovalent fragment comprising VL, VH, CL, and CH1 domains; (ii) a F(ab′)2 fragment, a bivalent fragment comprising two Fab fragments linked by a disulfide bridge at a hinge region; (iii) a Fv fragment comprising VH and CH1 domains; (iv) a Fv fragment comprising VL and VH domains of a single arm of an antibody, (v) a diabody (dAb) fragment comprising a VH domain; (vi) a camelid or camelized variable domain; (vii) a scFv, a fusion protein of VH and VL regions; or (viii) a single domain antibody. In some embodiments, an antibody or antigen-binding fragment thereof described herein comprises or is a heavy chain and a light chain (e.g., a half antibody).

In some embodiments, an antibody or antigen-binding fragment thereof described herein comprises: (a) a VH comprising one, two, or three VH CDR sequences in Table 1; and/or (b) a VL comprising one, two, or three VL CDR sequences in Table 1. In some embodiments, an antibody or antigen-binding fragment thereof described herein comprises: (a) a VH with at least about 80%, at least about 85%, at least about 90%, at least about 91%, at least about 92%, at least about 93%, at least about 94%, at least about 95%, or more identity to a VH in Table 1 (e.g., in areas outside of CDR regions); and/or (a) a VL with at least about 80%, at least about 85%, at least about 90%, at least about 91%, at least about 92%, at least about 93%, at least about 94%, at least about 95%, or more identity to a VL in Table 1 (e.g., in areas outside of CDR regions). In some embodiments, an antibody or antigen-binding fragment thereof described herein comprises: (a) a heavy chain with at least about 80%, at least about 85%, at least about 90%, at least about 91%, at least about 92%, at least about 93%, at least about 94%, at least about 95%, at least about 96%, at least about 97%, at least about 98%, at least about 99%, or more identity to a heavy chain in Table 1 (e.g., in areas outside of CDR regions); and/or (a) a light chain with at least about 80%, at least about 85%, at least about 90%, at least about 91%, at least about 92%, at least about 93%, at least about 94%, at least about 95%, at least about 96%, at least about 97%, at least about 98%, at least about 99%, or more identity to a light chain in Table 1 (e.g., in areas outside of CDR regions).

In some embodiments, an antibody or antigen-binding fragment thereof described herein comprises: (a) a VH comprising a VH CDR1 amino acid sequence of SEQ ID NO: 1, a VH CDR2 amino acid sequence of SEQ ID NO: 2, and a VH CDR3 amino acid sequence of SEQ ID NO: 3; and (b) a VL comprising a VL CDR1 amino acid sequence of SEQ ID NO: 11, a VL CDR2 amino acid sequence of SEQ ID NO: 12, and a VL CDR3 amino acid sequence of SEQ ID NO: 13. In some embodiments, an antibody or antigen-binding fragment thereof described herein comprises: (a) a VH comprising a VH CDR1 amino acid sequence of SEQ ID NO: 4, a VH CDR2 amino acid sequence of SEQ ID NO: 5, and a VH CDR3 amino acid sequence of SEQ ID NO: 6; and (b) a VL comprising a VL CDR1 amino acid sequence of SEQ ID NO: 14, a VL CDR2 amino acid sequence of SEQ ID NO: 15, and a VL CDR3 amino acid sequence of SEQ ID NO: 13. In some embodiments, an antibody or antigen-binding fragment thereof described herein comprises: (a) a VH comprising a VH CDR1 amino acid sequence of SEQ ID NO: 7, a VH CDR2 amino acid sequence of SEQ ID NO: 8, and a VH CDR3 amino acid sequence of SEQ ID NO: 6; and (b) a VL comprising a VL CDR1 amino acid sequence of SEQ ID NO: 14, a VL CDR2 amino acid sequence of SEQ ID NO: 15, and a VL CDR3 amino acid sequence of SEQ ID NO: 13.

In some embodiments, an antibody or antigen-binding fragment thereof described herein comprises a VH comprising an amino acid sequence of SEQ ID NO: 9, or an amino acid sequence at least about 80%, at least about 85%, at least about 90%, at least about 95%, at least about 99% identical or higher to SEQ ID NO: 9 (e.g., in areas outside of CDR regions). In some embodiments, an antibody or antigen-binding fragment thereof described herein comprises a VL comprising an amino acid sequence of SEQ ID NO: 16, or an amino acid sequence at least about 80%, at least about 85%, at least about 90%, at least about 95%, at least about 99% identical or higher to SEQ ID NO: 16 (e.g., in areas outside of CDR regions). In some embodiments, an antibody or antigen-binding fragment thereof described herein comprises a VH comprising an amino acid sequence of SEQ ID NO: 9 and a VL comprising an amino acid sequence of SEQ ID NO: 16.

In some embodiments, an antibody or antigen-binding fragment thereof described herein comprises a heavy chain comprising an amino acid sequence of SEQ ID NO: 10, or an amino acid sequence at least about 85%, at least about 90%, at least about 95%, at least about 99% identical or higher to SEQ ID NO: 10 (e.g., in areas outside of CDR regions). In some embodiments, an antibody or antigen-binding fragment thereof described herein comprises a light chain comprising an amino acid sequence of SEQ ID NO: 17, or an amino acid sequence at least about 85%, at least about 90%, at least about 95%, at least about 99% identical or higher to SEQ ID NO: 17 (e.g., in areas outside of CDR regions). In some embodiments, an antibody or antigen-binding fragment thereof described herein comprises a heavy chain comprising an amino acid sequence of SEQ ID NO: 10 and a light chain comprising an amino acid sequence of SEQ ID NO: 17.

In some embodiments, an antibody or antigen-binding fragment thereof described herein comprises: (a) a VH comprising a VH CDR1 amino acid sequence of SEQ ID NO: 18, a VH CDR2 amino acid sequence of SEQ ID NO: 19, and a VH CDR3 amino acid sequence of SEQ ID NO: 20; and (b) a VL comprising a VL CDR1 amino acid sequence of SEQ ID NO: 28, a VL CDR2 amino acid sequence of SEQ ID NO: 29, and a VL CDR3 amino acid sequence of SEQ ID NO: 30. In some embodiments, an antibody or antigen-binding fragment thereof described herein comprises: (a) a VH comprising a VH CDR1 amino acid sequence of SEQ ID NO: 21, a VH CDR2 amino acid sequence of SEQ ID NO: 22, and a VH CDR3 amino acid sequence of SEQ ID NO: 23; and (b) a VL comprising a VL CDR1 amino acid sequence of SEQ ID NO: 31, a VL CDR2 amino acid sequence of SEQ ID NO: 32, and a VL CDR3 amino acid sequence of SEQ ID NO: 30. In some embodiments, an antibody or antigen-binding fragment thereof described herein comprises: (a) a VH comprising a VH CDR1 amino acid sequence of SEQ ID NO: 24, a VH CDR2 amino acid sequence of SEQ ID NO: 25, and a VH CDR3 amino acid sequence of SEQ ID NO: 23; and (b) a VL comprising a VL CDR1 amino acid sequence of SEQ ID NO: 31, a VL CDR2 amino acid sequence of SEQ ID NO: 32, and a VL CDR3 amino acid sequence of SEQ ID NO: 30.

In some embodiments, an antibody or antigen-binding fragment thereof described herein comprises a VH comprising an amino acid sequence of SEQ ID NO: 26, or an amino acid sequence at least about 80%, at least about 85%, at least about 90%, at least about 95%, at least about 99% identical or higher to SEQ ID NO: 26 (e.g., in areas outside of CDR regions). In some embodiments, an antibody or antigen-binding fragment thereof described herein comprises a VL comprising an amino acid sequence of SEQ ID NO: 33, or an amino acid sequence at least about 80%, at least about 85%, at least about 90%, at least about 95%, at least about 99% identical or higher to SEQ ID NO: 33 (e.g., in areas outside of CDR regions). In some embodiments, an antibody or antigen-binding fragment thereof described herein comprises a VH comprising an amino acid sequence of SEQ ID NO: 26 and a VL comprising an amino acid sequence of SEQ ID NO: 33.

In some embodiments, an antibody or antigen-binding fragment thereof described herein comprises a heavy chain comprising an amino acid sequence of SEQ ID NO: 27, or an amino acid sequence at least about 85%, at least about 90%, at least about 95%, at least about 99% identical or higher to SEQ ID NO: 27 (e.g., in areas outside of CDR regions). In some embodiments, an antibody or antigen-binding fragment thereof described herein comprises a light chain comprising an amino acid sequence of SEQ ID NO: 34, or an amino acid sequence at least about 85%, at least about 90%, at least about 95%, at least about 99% identical or higher to SEQ ID NO: 34 (e.g., in areas outside of CDR regions). In some embodiments, an antibody or antigen-binding fragment thereof described herein comprises a heavy chain comprising an amino acid sequence of SEQ ID NO: 27 and a light chain comprising an amino acid sequence of SEQ ID NO: 34.

In some embodiments, an antibody or antigen-binding fragment thereof described herein comprises: (a) a VH comprising a VH CDR1 amino acid sequence of SEQ ID NO: 35, a VH CDR2 amino acid sequence of SEQ ID NO: 36, and a VH CDR3 amino acid sequence of SEQ ID NO: 37; and (b) a VL comprising a VL CDR1 amino acid sequence of SEQ ID NO: 44, a VL CDR2 amino acid sequence of SEQ ID NO: 45, and a VL CDR3 amino acid sequence of SEQ ID NO: 46. In some embodiments, an antibody or antigen-binding fragment thereof described herein comprises: (a) a VH comprising a VH CDR1 amino acid sequence of SEQ ID NO: 35, a VH CDR2 amino acid sequence of SEQ ID NO: 38, and a VH CDR3 amino acid sequence of SEQ ID NO: 39; and (b) a VL comprising a VL CDR1 amino acid sequence of SEQ ID NO: 47, a VL CDR2 amino acid sequence of SEQ ID NO: 48, and a VL CDR3 amino acid sequence of SEQ ID NO: 46. In some embodiments, an antibody or antigen-binding fragment thereof described herein comprises: (a) a VH comprising a VH CDR1 amino acid sequence of SEQ ID NO: 40, a VH CDR2 amino acid sequence of SEQ ID NO: 41, and a VH CDR3 amino acid sequence of SEQ ID NO: 39; and (b) a VL comprising a VL CDR1 amino acid sequence of SEQ ID NO: 47, a VL CDR2 amino acid sequence of SEQ ID NO: 48, and a VL CDR3 amino acid sequence of SEQ ID NO: 46.

In some embodiments, an antibody or antigen-binding fragment thereof described herein comprises a VH comprising an amino acid sequence of SEQ ID NO: 42, or an amino acid sequence at least about 80%, at least about 85%, at least about 90%, at least about 95%, at least about 99% identical or higher to SEQ ID NO: 42 (e.g., in areas outside of CDR regions). In some embodiments, an antibody or antigen-binding fragment thereof described herein comprises a VL comprising an amino acid sequence of SEQ ID NO: 49, or an amino acid sequence at least about 80%, at least about 85%, at least about 90%, at least about 95%, at least about 99% identical or higher to SEQ ID NO: 49 (e.g., in areas outside of CDR regions). In some embodiments, an antibody or antigen-binding fragment thereof described herein comprises a VH comprising an amino acid sequence of SEQ ID NO: 42 and a VL comprising an amino acid sequence of SEQ ID NO: 49.

In some embodiments, an antibody or antigen-binding fragment thereof described herein comprises a heavy chain comprising an amino acid sequence of SEQ ID NO: 43, or an amino acid sequence at least about 85%, at least about 90%, at least about 95%, at least about 99% identical or higher to SEQ ID NO: 43 (e.g., in areas outside of CDR regions). In some embodiments, an antibody or antigen-binding fragment thereof described herein comprises a light chain comprising an amino acid sequence of SEQ ID NO: 50, or an amino acid sequence at least about 85%, at least about 90%, at least about 95%, at least about 99% identical or higher to SEQ ID NO: 50 (e.g., in areas outside of CDR regions). In some embodiments, an antibody or antigen-binding fragment thereof described herein comprises a heavy chain comprising an amino acid sequence of SEQ ID NO: 43 and a light chain comprising an amino acid sequence of SEQ ID NO: 50.

In some embodiments, an antibody or antigen-binding fragment thereof described herein comprises a heavy chain comprising an amino acid sequence of SEQ ID NO: 51, or an amino acid sequence at least about 45% at least about 50% at least about 55%, at least about 60%, at least about 65%, at least about 70%, at least about 75%, at least about 80%, at least about 85%, at least about 90%, at least about 95%, at least about 99% identical or higher to SEQ ID NO: 51 (e.g., in areas outside of CDR regions). In some embodiments, an antibody or antigen-binding fragment thereof described herein comprises a light chain comprising an amino acid sequence of SEQ ID NO: 52, or an amino acid sequence at least about 85%, at least about 90%, at least about 95%, at least about 99% identical or higher to SEQ ID NO: 52 (e.g., in areas outside of CDR regions). In some embodiments, an antibody or antigen-binding fragment thereof described herein comprises a heavy chain comprising an amino acid sequence of SEQ ID NO: 51 and a light chain comprising an amino acid sequence of SEQ ID NO: 52.

In some embodiments, an antibody or antigen-binding fragment thereof described herein comprises a heavy chain comprising an amino acid sequence of SEQ ID NO: 53, or an amino acid sequence an at least about 45% at least about 50% at least about 55%, at least about 60%, at least about 65%, at least about 70%, at least about 75%, at least about 85%, at least about 90%, at least about 95%, at least about 99% identical or higher to SEQ ID NO: 53 (e.g., in areas outside of CDR regions). In some embodiments, an antibody or antigen-binding fragment thereof described herein comprises a light chain comprising an amino acid sequence of SEQ ID NO: 54, or an amino acid sequence at least about 85%, at least about 90%, at least about 95%, at least about 99% identical or higher to SEQ ID NO: 54 (e.g., in areas outside of CDR regions). In some embodiments, an antibody or antigen-binding fragment thereof described herein comprises a heavy chain comprising an amino acid sequence of SEQ ID NO: 53 and a light chain comprising an amino acid sequence of SEQ ID NO: 54.

In some embodiments, an antibody or antigen-binding fragment thereof described herein comprises a heavy chain comprising an amino acid sequence of SEQ ID NO: 55, or an amino acid sequence at least about 45% at least about 50% at least about 55%, at least about 60%, at least about 65%, at least about 70%, at least about 75%, at least about 85%, at least about 90%, at least about 95%, at least about 99% identical or higher to SEQ ID NO: 55 (e.g., in areas outside of CDR regions). In some embodiments, an antibody or antigen-binding fragment thereof described herein comprises a light chain comprising an amino acid sequence of SEQ ID NO: 56, or an amino acid sequence at least about 85%, at least about 90%, at least about 95%, at least about 99% identical or higher to SEQ ID NO: 56 (e.g., in areas outside of CDR regions). In some embodiments, an antibody or antigen-binding fragment thereof described herein comprises a heavy chain comprising an amino acid sequence of SEQ ID NO: 55 and a light chain comprising an amino acid sequence of SEQ ID NO: 56.

In some embodiments, an antibody or antigen-binding fragment thereof described herein comprises a heavy chain comprising an amino acid sequence of SEQ ID NO: 57, or an amino acid sequence at least about 45% at least about 50% at least about 55%, at least about 60%, at least about 65%, at least about 70%, at least about 75%, at least about 85%, at least about 90%, at least about 95%, at least about 99% identical or higher to SEQ ID NO: 57 (e.g., in areas outside of CDR regions). In some embodiments, an antibody or antigen-binding fragment thereof described herein comprises a light chain comprising an amino acid sequence of SEQ ID NO: 58, or an amino acid sequence at least about 85%, at least about 90%, at least about 95%, at least about 99% identical or higher to SEQ ID NO: 58 (e.g., in areas outside of CDR regions). In some embodiments, an antibody or antigen-binding fragment thereof described herein comprises a heavy chain comprising an amino acid sequence of SEQ ID NO: 57 and a light chain comprising an amino acid sequence of SEQ ID NO: 58.

In some embodiments, an antibody or antigen-binding fragment thereof described herein comprises a heavy chain comprising an amino acid sequence of SEQ ID NO: 59, or an amino acid sequence at least about 45% at least about 50% at least about 55%, at least about 60%, at least about 65%, at least about 70%, at least about 75%, at least about 85%, at least about 90%, at least about 95%, at least about 99% identical or higher to SEQ ID NO: 59 (e.g., in areas outside of CDR regions). In some embodiments, an antibody or antigen-binding fragment thereof described herein comprises a light chain comprising an amino acid sequence of SEQ ID NO: 60, or an amino acid sequence at least about 85%, at least about 90%, at least about 95%, at least about 99% identical or higher to SEQ ID NO: 60 (e.g., in areas outside of CDR regions). In some embodiments, an antibody or antigen-binding fragment thereof described herein comprises a heavy chain comprising an amino acid sequence of SEQ ID NO: 59 and a light chain comprising an amino acid sequence of SEQ ID NO: 60.

In some embodiments, an antibody or antigen-binding fragment thereof provided herein comprises an Fc region. In some embodiments, an Fc region comprising a modification has reduced effector function relative to a reference Fc region without a modification. In some embodiments, a modification comprises one or more of the following amino acid substitutions: M252Y, S254T, T256E, N297A, L234A, L235A, C220, C226S, C229S, P238S, C226S, C229S, E233P, L234V, L235A, G237A, L234F, N297G, L235E, P329A, P329G, P331S, M428L, N434S, or combinations thereof, numbering according to EU index. In some embodiments, a modification comprises L234A, L235A, and P329A, numbering according to EU index.

TABLE 1
Amino acid sequences of exemplary payload neutralizing antibodies and
antigen-binding fragments thereof.

| | | |
|---|---|---|
| Description | Sequence | SEQ ID NO |

Exemplary Payload Neutralizing Antibody 1

| | | |
|---|---|---|
| VHCDR1 | GFTFSDYA | 1 |
| (IMGT) | | |
| VHCDR2 | ISSTGSYK | 2 |
| (IMGT) | | |
| VHCDR3 | LASVITNDYFEY | 3 |
| (IMGT) | | |
| VHCDR1 | GFTFSDY | 4 |
| (Chothia) | | |
| VHCDR2 | SSTGSY | 5 |
| (Chothia) | | |
| VHCDR3 | SVITNDYFEY | 6 |
| (Chothia/ | | |
| Kabat) | | |
| VHCDR1 | DYAMS | 7 |
| (Kabat) | | |
| VHCDR2 | TISSTGSYKYYLDSVKG | 8 |
| (Kabat) | | |
| VH | EVQLVESGGGLVKPGGSLKLSCAASGFTFSDYAMSWFRQ | 9 |
| | APEKRLEWVATISSTGSYKYYLDSVKGRFTISRDNSKNTL | |
| | YLQMSSLRSEDTAVYYCLASVITNDYFEYWGQGTLVTVSS | |
| Heavy chain | EVQLVESGGGLVKPGGSLKLSCAASGFTFSDYAMSWFRQ | 10 |
| | APEKRLEWVATISSTGSYKYYLDSVKGRFTISRDNSKNTL | |
| | YLQMSSLRSEDTAVYYCLASVITNDYFEYWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGTQ | |
| | TYICNVNHKPSNTKVDKRVEPKSCDKTHTCPPCPAPEAA | |
| | GGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALAAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPGK | |
| VLCDR1 | QDVGTA | 11 |
| (IMGT) | | |
| VLCDR2 | WAS | 12 |
| (IMGT) | | |
| VLCDR3 | QQYANLPYT | 13 |
| (IMGT/ | | |
| Chothia/ | | |
| Kabat) | | |
| VLCDR1 | RASQDVGTAVA | 14 |
| (Chothia/ | | |
| Kabat) | | |
| VLCDR2 | WASTRHT | 15 |
| (Chothia/ | | |
| Kabat) | | |
| VL | DIVMTQSPSSLSASVGDRVTITCRASQDVGTAVAWYQQK | 16 |
| | PGQSPKLLIYWASTRHTGVPDRFSGSGSGTDFTLTISGLQS | |
| | EDEADYFCQQYANLPYTFGGGTKLEIK | |
| Light chain | DIVMTQSPSSLSASVGDRVTITCRASQDVGTAVAWYQQK | 17 |
| | PGQSPKLLIYWASTRHTGVPDRFSGSGSGTDFTLTISGLQS | |
| | EDEADYFCQQYANLPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Exemplary Payload Neutralizing Antibody 2

| | | |
|---|---|---|
| VHCDR1 | GFTFSDYA | 18 |
| (IMGT) | | |
| VHCDR2 | ISSTGSYS | 19 |
| (IMGT) | | |
| VHCDR3 | LASSYTNDYFEY | 20 |
| (IMGT) | | |
| VHCDR1 | GFTFSDY | 21 |
| (Chothia) | | |
| VHCDR2 | SSTGSY | 22 |
| (Chothia) | | |
| VHCDR3 | SSYTNDYFEY | 23 |
| (Chothia/ | | |
| Kabat) | | |
| VHCDR1 | DYAMS | 24 |
| (Kabat) | | |
| VHCDR2 | TISSTGSYSYYLDSVKG | 25 |
| (Kabat) | | |
| VH | EVQLVESGGGLVKPGGSLKLSCAASGFTFSDYAMSWFRQ | 26 |
| | APEKRLEWVATISSTGSYSYYLDSVKGRFTISRDNSKNTL | |
| | YLQMSSLRSEDTAVYYCLASSYTNDYFEYWGQGTLVTVSS | |
| Heavy chain | EVQLVESGGGLVKPGGSLKLSCAASGFTFSDYAMSWFRQ | 27 |
| | APEKRLEWVATISSTGSYSYYLDSVKGRFTISRDNSKNTL | |
| | YLQMSSLRSEDTAVYYCLASSYTNDYFEYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKTHTCPPCPAPEA | |
| | AGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALAAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPGK | |
| VLCDR1 | QDVGTA | 28 |
| (IMGT) | | |
| VLCDR2 | WAS | 29 |
| (IMGT) | | |
| VLCDR3 | QQYANLPYT | 30 |
| (IMGT/ | | |
| Chothia/ | | |
| Kabat) | | |
| VLCDR1 | RASQDVGTAVA | 31 |
| (Chothia/ | | |
| Kabat) | | |
| VLCDR2 | WASTRHT | 32 |
| (Chothia/ | | |
| Kabat) | | |
| VL | DIVMTQSPSSLSASVGDRVTITCRASQDVGTAVAWYQQK | 33 |
| | PGQSPKLLIYWASTRHTGVPDRFSGSGSGTDFTLTISGLQS | |
| | EDEADYFCQQYANLPYTFGGGTKLEIK | |
| Light chain | DIVMTQSPSSLSASVGDRVTITCRASQDVGTAVAWYQQK | 34 |
| | PGQSPKLLIYWASTRHTGVPDRFSGSGSGTDFTLTISGLQS | |
| | EDEADYFCQQYANLPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Exemplary Payload Neutralizing Antibody 3

| | | |
|---|---|---|
| VHCDR1 | GFTFSGYT | 35 |
| (IMGT/ | | |
| Chothia) | | |
| VHCDR2 | ISSAGSYI | 36 |
| (IMGT) | | |
| VHCDR3 | LASYITTSYFEH | 37 |
| (IMGT) | | |
| VHCDR2 | SSAGSY | 38 |
| (Chothia) | | |
| VHCDR3 | SYITTSYFEH | 39 |
| (Chothia/ | | |
| Kabat) | | |
| VHCDR1 | GYTMS | 40 |
| (Kabat) | | |
| VHCDR2 | TISSAGSYIYYLDSVKG | 41 |
| (Kabat) | | |
| VH | EVQLVESGGGLVKPGGSLKLSCAASGFTFSGYTMSWFRQ | 42 |
| | APEKRLEWVATISSAGSYIYYLDSVKGRFTISRDNSKNTL | |
| | YLQMSSLRSEDTAVYYCLASYITTSYFEHWGQGTLVTVSS | |
| Heavy chain | EVQLVESGGGLVKPGGSLKLSCAASGFTFSGYTMSWFRQ | 43 |
| | APEKRLEWVATISSAGSYIYYLDSVKGRFTISRDNSKNTL | |
| | YLQMSSLRSEDTAVYYCLASYITTSYFEHWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGTQ | |
| | TYICNVNHKPSNTKVDKRVEPKSCDKTHTCPPCPAPEAA | |
| | GGPSVFLFPPKPKDTLMISRTPEVTCVVVDVSHEDPEVKF | |
| | NWYVDGVEVHNAKTKPREEQYNSTYRVVSVLTVLHQD | |
| | WLNGKEYKCKVSNKALAAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPGK | |
| VLCDR1 | QDVGTA | 44 |
| (IMGT) | | |
| VLCDR2 | WAS | 45 |
| (IMGT) | | |
| VLCDR3 | QQYSNLPYT | 46 |
| (IMGT/ | | |
| Chothia/ | | |
| Kabat) | | |
| VLCDR1 | RASQDVGTAVA | 47 |
| (Chothia/ | | |
| Kabat) | | |
| VLCDR2 | WASTRHT | 48 |
| (Chothia/ | | |
| Kabat) | | |
| VL | DIVMTQSPSSLSASVGDRVTITCRASQDVGTAVAWYQQK | 49 |
| | PGQSPKLLIYWASTRHTGVPDRFSGSGSGTDFTLTISGLQS | |
| | EDEADYFCQQYSNLPYTFGGGTKLEIK | |
| Light chain | DIVMTQSPSSLSASVGDRVTITCRASQDVGTAVAWYQQK | 50 |
| | PGQSPKLLIYWASTRHTGVPDRFSGSGSGTDFTLTISGLQS | |
| | EDEADYFCQQYSNLPYTFGGGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

Reference Antibody Fragment 1

| | | |
|---|---|---|
| Heavy chain | EVQLVESGGGLVKPGGSLKLSCAASGFTFSGYAMSWFRQ | 51 |
| | APEKRLEWVATISSGGSYTYYLDSVKGRFTISRDNSKNTL | |
| | YLQMSSLRSEDTAVYYCLASLITTDYFEYWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGTQ | |
| | TYICNVNHKPSNTKVDKRVEPKSCDKT | |
| Light chain | DIVMTQSPSSLSASVGDRVTITCRASQDVGTAVAWYQQK | 52 |
| | PGQSPKLLIYWASTRHTGVPDRFSGSGSGTDFTLTISGLQS | |
| | EDEADYFCQQYSNYPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Reference Antibody Fragment 2

| | | |
|---|---|---|
| Heavy chain | EVQLVESGGGLVKPGGSLKLSCAASGLTFSGYAMSWFR | 53 |
| | QAPEKRLEWVATISSGGSYTYYLDSVKGRFTISRDNSKNT | |
| | LYLQMSSLRSEDTAVYYCLASLITTDYFEYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKT | |
| Light chain | DIVMTQSPSSLSASVGDRVTITCRASQDVGTAVAWYQQK | 54 |
| | PGQSPKLLIYWASTRYTGVPDRFSGSGSGTDFTLTISGLQS | |
| | EDEADYFCQQYSNYPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Exemplary Payload Neutralizing Antibody Fragment 1

| | | |
|---|---|---|
| Heavy chain | EVQLVESGGGLVKPGGSLKLSCAASGFTFSDYAMSWFRQ | 55 |
| | APEKRLEWVATISSTGSYKYYLDSVKGRFTISRDNSKNTL | |
| | YLQMSSLRSEDTAVYYCLASVITNDYFEYWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGTQ | |
| | TYICNVNHKPSNTKVDKRVEPKSCDKT | |
| Light chain | DIVMTQSPSSLSASVGDRVTITCRASQDVGTAVAWYQQK | 56 |
| | PGQSPKLLIYWASTRHTGVPDRFSGSGSGTDFTLTISGLQS | |
| | EDEADYFCQQYANLPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Exemplary Payload Neutralizing Antibody Fragment 2

| | | |
|---|---|---|
| Heavy chain | EVQLVESGGGLVKPGGSLKLSCAASGFTFSDYAMSWFRQ | 57 |
| | APEKRLEWVATISSTGSYSYYLDSVKGRFTISRDNSKNTL | |
| | YLQMSSLRSEDTAVYYCLASSYTNDYFEYWGQGTLVTV | |
| | SSASTKGPSVFPLAPSSKSTSGGTAALGCLVKDYFPEPVT | |
| | VSWNSGALTSGVHTFPAVLQSSGLYSLSSVVTVPSSSLGT | |
| | QTYICNVNHKPSNTKVDKRVEPKSCDKT | |
| Light chain | DIVMTQSPSSLSASVGDRVTITCRASQDVGTAVAWYQQK | 58 |
| | PGQSPKLLIYWASTRHTGVPDRFSGSGSGTDFTLTISGLQS | |
| | EDEADYFCQQYANLPYTFGGGTKLEIKRTVAAPSVFIFPP | |
| | SDEQLKSGTASVVCLLNNFYPREAKVQWKVDNALQSGN | |
| | SQESVTEQDSKDSTYSLSSTLTLSKADYEKHKVYACEVT | |
| | HQGLSSPVTKSFNRGEC | |

Exemplary Payload Neutralizing Antibody Fragment 3

| | | |
|---|---|---|
| Heavy chain | EVQLVESGGGLVKPGGSLKLSCAASGFTFSGYTMSWFRQ | 59 |
| | APEKRLEWVATISSAGSYIYYLDSVKGRFTISRDNSKNTL | |
| | YLQMSSLRSEDTAVYYCLASYITTSYFEHWGQGTLVTVS | |
| | SASTKGPSVFPLAPSSKSTSGGTAALGCLVKDYFPEPVTV | |
| | SWNSGALTSGVHTFPAVLQSSGLYSLSSVVTVPSSSLGTQ | |
| | TYICNVNHKPSNTKVDKRVEPKSCDKT | |
| Light chain | DIVMTQSPSSLSASVGDRVTITCRASQDVGTAVAWYQQK | 60 |
| | PGQSPKLLIYWASTRHTGVPDRFSGSGSGTDFTLTISGLQS | |
| | EDEADYFCQQYSNLPYTFGGGTKLEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

### Fc Mutations

In some embodiments, an antibody or antigen-binding fragment thereof provided herein comprises an Fc region. In some embodiments, the Fc region comprises at least one modification, e.g., at least one mutation that reduces effector function. In some embodiments, the Fc region comprises a modification that reduces CDC, ADCC or both. In some embodiments, the Fc region comprises at least one modification, e.g., at least one mutation that increases effector function. In some embodiments, the Fc region comprises at least one modification that increases CDC, ADCC or both. In some embodiments, the Fc region comprises at least one modification, e.g., at least one mutation that increases antibody stability. In some embodiments, stability is half-life. In some embodiments, half-life is circulation half-life. In some embodiments, half-life is half-life in blood. In some embodiments, blood is serum.

Examples of such mutations can be found in Saunders et al., 2019, Front. Immunol. 10:1296, which is hereby incorporated by reference in its entirety. Table 1 of Saunders provides Fc modifications that increase antibody effector function. Table 2 of Saunders provides Fc modifications that improve antibody circulation half-life. Table 3 of Saunders provides Fc modifications that reduce antibody effector function. In some embodiments, an Fc region is a human IgG1 Fc region that comprises one or more of the following mutations: M252Y, S254T, T256E, N297A, L234A, L235A, C226S, C229S, P238S, E233P, L234V, G237A, L234F, N297G, L235E, P329A, P329G, P331S, M428L, N434S, L238R, G236R, D265A, K322A, L235Q, K322Q, H268A, A330S, A327G, AG236, L235G, or combinations thereof, numbering according to EU index. In some embodiments, an Fc modification comprises L234A and L235A, numbering according to EU index. In some embodiments, an Fc modification comprises L234A, L235A, and P329A, numbering according to EU index. In some embodiments, an Fc modification comprises L234A, L235A, and P329G, numbering according to EU index. In some embodiments, an Fc modification comprises M252Y, S254T, and T256E, numbering according to EU index. In some embodiments, an Fc modification comprises M428L and N434S, numbering according to EU index. In some embodiments, an Fc modification comprises L234A, L235A, P329A, M252Y, S254T, and T256E, numbering according to EU index. In some embodiments, an Fc modification comprises L234A, L235A, P329G, M252Y, S254T, and T256E, numbering according to EU index. In some embodiments, an Fc modification comprises L234A, L235A, P329G, M428L and N434S, numbering according to EU index. In some embodiments, an Fc modification comprises L234A, L235A, P329A, M428L and N434S, numbering according to EU index. These and other exemplary Fc modifications are described in more detail below.

Fc Modifications that Reduce Effector Function

In some embodiments, the Fc modification, e.g., mutation reduces effector function. In some embodiments, effector function comprises ADCC, CDC or both. In some embodiments, reduced effector function comprises reduced cytotoxicity. In some embodiments, reduces is abolishes. In some embodiments, an Fc is from IgG1 or IgG3 and the mutation reduces effector function. In some embodiments, an Fc is from IgG1 and comprises at least one mutation that reduces effector function. It will be known by a skilled artisan that IgG2 and IgG4 possess reduced effector function and are not generally cytotoxic in nature. Additionally, mutations such as S228P and L235E in IgG4 are known to reduce effector function even more.

In some embodiments, the Fc region (e.g., from human IgG1) comprises at least one mutation that reduces effector function. In some embodiments, a mutation is a mutation that decreases binding to an Fc receptor. In some embodiments, a mutation is a mutation that decreases binding to C1q. In some embodiments, a mutation that decreases binding to Fc receptor decreases ADCC. In some embodiments, at least one mutation that reduces effector function includes L234A and L235A within IgG1 (also termed LALA), numbering according to EU index. In some embodiments, a LALA mutation is combined with the P329G mutation (also termed LALAPG), numbering according to EU index. In some embodiments, a LALA mutation is combined with the P329A mutation (also termed LALAPA), numbering according to EU index. In some embodiments, a mutation is mutation of N297 within IgG1, numbering according to EU index. As N-glycans are linked to N297, its mutation abrogates the glycosylation of this residue. In some embodiments, mutation of N297 is mutation to alanine (N297A). In some embodiments, mutation of N297 is mutation to glutamine (N297Q). In some embodiments, mutation of N297 is mutation to glycine (N297G). In some embodiments, a mutation is a plurality of mutations that decrease binding to an Fc receptor. In some embodiments, a plurality comprises or consists of G236R and L328R within IgG1, numbering according to EU index. In some embodiments, a plurality comprises or consists of S298G and T299A within IgG1, numbering according to EU index. In some embodiments, a plurality comprises or consists of L234F, L235E and D265A within IgG1, numbering according to EU index. In some embodiments, a plurality comprises or consists of L234F, L235E and P331S within IgG1, numbering according to EU index. In some embodiments, a plurality comprises or consists of L235A, G237A and E318A within IgG1, numbering according to EU index.

In some embodiments, a plurality comprises or consists of L234A and G237A within IgG1 (also termed LAGA), numbering according to EU index. In some embodiments, a plurality comprises or consists of L234A and L235E within IgG1 (also termed LALE), numbering according to EU index. In some embodiments, a plurality comprises or consists of D265A and P329A within IgG1 (also termed DAPA), numbering according to EU index. In some embodiments, a plurality comprises or consists of L234A, L235A, and K322A within IgG1 (also termed LALAKA), numbering according to EU index. In some embodiments, a plurality comprises or consists of L234F, L235Q, and K322Q within IgG1 (also termed FQQ), numbering according to EU index. In some embodiments, a plurality comprises or consists of L234A, L235A, G237A, P238S, H268A, A330S, and P331S within IgG1, numbering according to EU index. In some embodiments, a plurality comprises or consists of E233P, L234V, L235A, AG236, A327G, A330S, and P331S within IgG1 (also termed PVA-GSS), numbering according to EU index. In some embodiments, a plurality comprises or consists of L235G and G236R within IgG1 (also termed LGGR), numbering according to EU index.

In some embodiments, a modification is removal of glycosylation. In some embodiments, Fc glycosylation is removed enzymatically. In some embodiments, enzymatic de-glycosylation is performed with a deglycosylase. In some embodiments, enzymatic de-glycosylation is performed with a cleavase that cleaves sugars. Examples of enzymes for de-glycosylation include but are not limited to Peptide-N-Glycosidase F (PNGase) and Endoglycosidase H (Endo H).

Fc Modifications that Increase Effector Function

In some embodiments, an Fc modification, e.g., mutation increases effector function. In some embodiments, effector function comprises ADCC, CDC or both. In some embodiments, increased effector function comprises increased cytotoxicity. In some embodiments, the Fc is from IgG1 or IgG3 and the mutation increases effector function. In some embodiments, an Fc is from IgG1 and comprises at least one mutation that increases effector function.

In some embodiments, a mutation is a mutation that increases binding to an Fc receptor. In some embodiments, an Fc receptor is selected from FcγRI, FcγRIIA, FcγRIIB, FcγRIIIA, and FcγRIIIB. In some embodiments, a mutation is S267E within IgG1, numbering according to EU index. In some embodiments, a mutation is P238D within IgG1, numbering according to EU index. In some embodiments, a mutation is a plurality of mutations that increase binding to an Fc receptor. In some embodiments, a plurality comprises or consists of S267E and L328F within IgG1 (also termed SELF), numbering according to EU index. In some embodiments, a plurality comprises or consists of S267E, H268F and S324T within IgG1 (also termed EFT), numbering according to EU index. In some embodiments, a plurality comprises or consists of G237D, P238D, P271G and A330R within IgG1 (also termed V9), numbering according to EU index. In some embodiments, a plurality comprises or consists of G237D, P238D, H268D, P271G and A330R within IgG1 (also termed V11), numbering according to EU index. In some embodiments, a plurality comprises or consists of E233D, G237D, P238D, H268D, P271G and A330R within IgG1 (also termed V12), numbering according to EU index. The S267E mutation was found to enhance affinity toward the inhibitory FcγRIIB and also toward the activating FcγRIIA. The SELF mutations resulted in a substantial 430-fold increase in the binding toward FcγRIIB, with minimal alterations in binding to FcγRI and FcγRIIA-H131 in comparison to wild-type IgG1. The EFT mutation was found to increase FcγRIIB binding by 18-fold in comparison to wild-type IgG1. EFT also increased CDC, ADCC and antibody-dependent cellular phagocytosis (ADCP) activity. In some embodiments, a mutation that increases ADCC is the EFT plurality of mutations. P238D demonstrated enhanced binding to FcγRIIB with about 4.3-fold increased affinity in comparison to wild-type IgG1. P238D also significantly reduces the binding toward all other activating Fcγ receptors. V9 significantly enhanced the affinity of antibodies toward FcγRIIB, by approximately a 32-fold change in comparison to wild-type IgG1. V9 was also found to reduce the affinity toward FcγRIIA-R131 by about 3-fold in comparison to wild-type IgG1. VI I was found to enhance the affinity of antibodies for FcγRIIB by approximately 96-fold, while reducing the affinity toward FcγRIIA-R131 by about 3-fold in comparison to wild-type IgG1. V12 demonstrated significant enhancement of binding toward FcγRIIB, with 217-fold change in comparison to wild-type IgG1. V12 mutations also show no detectable binding toward FcγRIIIA allotypes, reduced FcγRI binding (0.061-fold change relative to wild-type IgG1) and FcγRIIA-H131 (0.068-fold change relative to wild-type IgG1). V12 slightly improves the binding toward FcγRIIA-R131, with a 2-fold binding increase in compared to wild-type IgG1.

In some embodiments, a mutation is a mutation that increases ADCC. In some embodiments, a mutation that increases ADCC is a plurality of mutations that increase ADCC. In some embodiments, a plurality of mutations comprises L235V, F243L, R292P, Y300L and P396L within IgG1, numbering according to EU index. In some embodiments, a plurality of mutations comprises S239D and 1332E within IgG1, numbering according to EU index. In some embodiments, S239D/1332E mutations also increase ADCP. In some embodiments, a plurality of mutations comprises S239D, A330L and 1332E within IgG1, numbering according to EU index. In some embodiments, S239D/A330L/1332E mutations also increase ADCP. In some embodiments, a plurality of mutations comprises G236A, A330L and I332E within IgG1, numbering according to EU index. In some embodiments, a plurality of mutations comprises S298A, E333A, and K334A within IgG1, numbering according to EU index. In some embodiments, a plurality of mutations comprises P247I and A339Q within IgG1, numbering according to EU index. In some embodiments, a plurality of mutations comprises G236A, S239D and 1332E within IgG1, numbering according to EU index. In some embodiments, G236A/S239D/1332E mutations also increase ADCP. In some embodiments, a plurality of mutations comprises L234Y, L235Q, G236W, S239M, H268D, D270E and S298A within a first heavy chain of IgG1 and D270E, K326D, A330M and K334E within the second heavy chain of IgG1, numbering according to EU index.

In some embodiments, an Fc is modified to increase ADCC by removal of fucosylation. In some embodiments, Fc fucosylation is removed enzymatically. In some embodiments, an Fc is afucosylated. In some embodiments, the method comprises performing afucosylation of an antibody or antigen-binding fragment thereof provided herein comprises an Fc region. In some embodiments, the antibody or antigen-binding fragment thereof are produced in a cell line engineered to produce afucosylated molecules.

In some embodiments, a mutation is a mutation that increases CDC. In some embodiments, a mutation that increases CDC is a plurality of mutations that increase CDC. In some embodiments, a plurality of mutations comprises G236A, S267E, H268F, S324T and I332E within IgG1, numbering according to EU index. In some embodiments, a plurality of mutation comprises K326W and E333S within IgG1, numbering according to EU index. In some embodiments, a plurality of mutation comprises E345R, E430G and S440Y within IgG1, numbering according to EU index.

Fc Modifications that Increase Stability

In some embodiments, an Fc region comprises at least one modification, e.g., at least one mutation that increases antibody stability. In some embodiments, stability is half-life. In some embodiments, half-life is circulation half-life. In some embodiments, half-life is half-life in blood. In some embodiments, blood is serum. In some embodiments, an Fc region comprises at least one mutation that increases the half-life of the antibody or antigen-binding fragment thereof. In some embodiments, a mutation that increases half-life is a mutation that increases binding to the neonatal Fc receptor (FcRn). In some embodiments, a mutation is N434H, V308P, and/or H435A within IgG1, numbering according to EU index. In some embodiments, a mutation that increases binding to FcRn is a plurality of mutations. In some embodiments, a plurality comprises or consists of M252Y, S254T and T256E within IgG1 (also termed YTE), numbering according to EU index. In some embodiments, a plurality comprises or consists of M428L and N434S within IgG1 (also termed LS), numbering according to EU index. In some embodiments, a plurality comprises or consists of M428L and N434A within IgG1 (also termed LA), numbering according to EU index. In some embodiments, a plurality comprises or consists of T250Q and M428L within IgG1 (also termed QL), numbering according to EU index. In some embodiments, a plurality comprises or consists of H433K and N434F within IgG1, numbering according to EU index. In some embodiments, a plurality comprises or consists of M252Y, S254T, T256E, H433K and N434F within IgG1, numbering according to EU index. In some embodiments, a plurality comprises or consists of T307A, E380A and N434A within IgG1, numbering according to EU index. In some embodiments, a plurality comprises or consists of M252Y, V308P and N343Y within IgG1, numbering according to EU index. In some embodiments, a plurality comprises or consists of mutation of H258D, T307Q and A378V within IgG1, numbering according to EU index. In some embodiments, a plurality comprises or consists of mutation of L309D, Q311H and N434S within IgG1, numbering according to EU index. In some embodiments, a plurality comprises or consists of I253A, H435A and H436A within IgG1, numbering according to EU index. In some embodiments, a plurality comprises or consists of I253A, H310A and H435A within IgG1, numbering according to EU index.

### Methods of Making

The present disclosure, among other things, provides methods of making antibodies or antigen-binding fragments thereof that bind specifically to a free drug (e.g., MMAE) of an antibody drug conjugate (ADC) described herein. In some embodiments, an antibody or antigen-binding fragment thereof described herein is identified using a display technology, such as yeast display, phage display, or ribosome display. In some embodiments, an antibody or antigen-binding fragment thereof described herein is identified using a hybridoma library (e.g., a mammalian hybridoma library, e.g., a mouse hybridoma library), followed by supernatant screening.

Combinatorial methods for generating antibodies or antigen-binding fragments thereof are described in, for example, U.S. Pat. No. 5,223,409; International Publication No. WO 92/18619; International Publication No. WO 91/17271; International Publication WO 92/20791; International Publication No. WO 92/15679; International Publication WO 93/01288; International Publication No. WO 92/01047; International Publication No. WO 92/09690; International Publication No. WO 90/02809; Fuchs et al., 1991, Bio/Technology. 9:1370-1372; Hay et al., 1992, Hum. Antibody Hybridomas. 3:81-85; Huse et al., 1989, Science. 246:1275-1281; Griffths et al., 1993, EMBO. J. 12:725-734; Hawkins et al., 1992, J. Mol. Biol. 226:889-896; Clackson et al., 1991, Nature. 352:624-628; Gram et al., 1992, PNAS. 89:3576-3580; Garrad et al., 1991, Bio/Technology. 9:1373-1377; Hoogenboom et al., 1991, Nuc. Acid. Res. 19:4133-4137; and Barbas et al., 1991, PNAS. 88:7978-7982, each of which his hereby incorporated by reference in its entirety.

An antibody or antigen-binding fragment thereof described herein can be derived from other species. A humanized antibody is an antibody produced by recombinant DNA technology, in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary, one or more residues in human FRs can be changed to residues at corresponding positions in a murine antibody so as to preserve binding affinity of the humanized antibody to a target. This change is sometimes called “back mutation.” Similarly, forward mutations may be made to revert back to murine sequence for a desired reason, e.g., stability or affinity to a target. Humanized antibodies generally are less likely to elicit an immune response in humans as compared to chimeric human antibodies because the former contain considerably fewer non-human components.

There are various methods for humanizing non-human antibodies. Suitable methods for making humanized antibodies in accordance with various aspects of innovations described herein are described in, e.g., EP U.S. Pat. No. 239,400; Jones et al., Nature 321:522-525 (1986); Riechmann et al., Nature 332:323-327 (1988); Verhoeyen et al., Science 239:1534-1536 (1988); Queen et al., Proc. Nat. Acad. ScL USA 86:10029 (1989); U.S. Pat. No. 6,180,370; and Orlandi et al., Proc. Natl. Acad. Sd. USA 86:3833 (1989); each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γl for CH and κ for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

An antibody or antigen-binding fragment thereof described herein can be or comprise a human antibody or antigen-binding fragment thereof. Completely human antibodies may be particularly desirable for therapeutic treatment of human subjects. Human antibodies can be made by a variety of methods including phage display methods described above using antibody libraries derived from human immunoglobulin sequences (see, e.g., U.S. Pat. Nos. 4,444,887 and 4,716,111; and International Publication Nos. WO 98/46645, WO 98/60433, WO 98/24893, WO 98/16664, WO 96/34096, WO 96/33735, and WO 91/10741; each of which is incorporated herein by reference in its entirety). Techniques are also available for the preparation of human monoclonal antibodies in, e.g., Cole et al., 1985, “Monoclonal Antibodies and Cancer Therapy,” Alan R. Riss; and Boerner et al., 1991, J. Immunol. 147 (1): 86-95, each of which is incorporated herein by reference in its entirety.

An antibody or antigen-binding fragment thereof described herein can be or comprise a chimeric antibody or antigen-binding fragment thereof. Illustrative methods of making chimeric antibodies are described, for example, in U.S. Pat. No. 4,816,567; and Morrison et al., 1984, PNAS. 81:6851-6855, each of which is incorporated by reference in its entirety. In some embodiments, a chimeric antibody is made by using recombinant techniques to combine a non-human variable region (e.g., a variable region derived from a mouse, rat, hamster, rabbit, or non-human primate, such as a monkey) with a human constant region.

Any suitable method can be used to introduce variability into one or more polynucleotide sequences encoding an antibody or antigen-binding fragment thereof described herein, including error-prone PCR, chain shuffling, and oligonucleotide-directed mutagenesis such as trinucleotide-directed mutagenesis (TRIM). In some embodiments, several CDR residues (e.g., 2-20 residues at a time) are randomized. CDR residues involved in antigen binding may be specifically identified, for example, using alanine scanning mutagenesis or modeling. CDR-H3 and CDR-L3, in particular, are often targeted for mutation. Introduction of diversity into variable regions and/or CDRs can be used to produce a secondary library. A secondary library can then be screened to identify antibody variants with improved affinity. Affinity maturation by constructing and reselecting from secondary libraries has been described, for example, in Hoogenboom et al., 2001, Methods in Molecular Biology. 178:1-37, incorporated by reference in its entirety.

### Polynucleotides

Provided herein, among other things, are polynucleotides encoding antibodies or antigen-binding fragments thereof described herein. In some embodiments, a nucleic acid encodes an antibody or antigen-binding fragment thereof that binds specifically to a free drug (e.g., MMAE) of an antibody drug conjugate (ADC). Provided herein are nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g., a mammalian cell for production of an antibody or antigen-binding fragment thereof described herein.

Nucleic acids encoding antibodies or antigen-binding fragments thereof described herein may be modified to include codons that are optimized for expression in a particular cell type or organism. Codon optimized sequences are synthetic sequences, and preferably encode an identical polypeptide (or biologically active fragment of a full-length polypeptide which has substantially the same activity as the full-length polypeptide) encoded by a non-codon optimized parent polynucleotide. In some embodiments, a coding region of a nucleic acid encoding an antibody or antigen-binding fragment thereof described herein, in whole or in part, may include an altered sequence to optimize codon usage for a particular cell type (e.g., a eukaryotic or prokaryotic cell). For example, a coding sequence for a humanized heavy or light chain variable region as described herein may be optimized for expression in a bacterial cell. Alternatively, a coding sequence may be optimized for expression in a mammalian cell (e.g., a CHO cell). Such a sequence may be described as a codon-optimized sequence.

Nucleic acid constructs described herein may be inserted into an expression vector or viral vector by methods known to the art, and nucleic acids may be operably linked to an expression control sequence. A vector comprising any nucleic acids or fragments thereof described herein is further provided herein. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, 1987, Vol. 153, Wu and Grossman, eds., Academic Press).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). Bacteriophage vectors, such as λGT10, λGT11, λZapII (Stratagene), λEMBL4, and λNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding an antibody or antigen-binding fragment thereof described herein, or to improve introduction of a nucleic acid into a cell. Use of cloning vectors, expression vectors, adapters, and linkers have been described (see, e.g., Sambrook et al., 1989, “Molecular Cloning, a Laboratory Manual,” 2^{nd} edition, Cold Spring Harbor Press, Cold Spring Harbor, N.Y.; and Ausubel et al., 1994, “Current Protocols in Molecular Biology,” Greene Publishing Associates and John Wiley & Sons, New York, N.Y., each of which is hereby incorporated by reference in its entirety).

In some embodiments, nucleic acids and vectors described herein are isolated and/or purified. Provided herein, among other things, is a composition comprising an isolated or purified nucleic acid, optionally in the form of a vector. Isolated nucleic acids and vectors may be prepared using standard techniques including, for example, alkali/SDS treatment, CsCl binding, column chromatography, agarose gel electrophoresis, and/or other techniques.

Any method known to one skilled in the art for insertion of nucleic acids into a vector may be used to construct expression vectors encoding an antibody or antigen-binding fragment thereof described herein under control of transcriptional and/or translational control signals. These methods may include in vitro recombinant DNA and synthetic techniques and in vivo recombination (see, e.g., Ausubel, supra or Sambrook, supra).

### Methods of Use

Provided herein, among other things, are methods of treating or preventing off-target effects of a free drug of an antibody drug conjugate (ADC) in a subject comprising administering an antibody or antigen-binding fragment thereof described herein to the subject. In some embodiments, an antibody or antigen-binding fragment thereof described herein binds specifically to a free drug of an ADC. Pharmaceutical compositions comprising an antibody or antigen-binding fragment thereof described herein can be for use in the manufacture of a medicament for treating or preventing off-target effects of a free drug of an ADC in a subject. In some embodiments, methods of neutralizing off-target effects of a free drug of an ADC in a subject comprise administering an antibody or antigen-binding fragment thereof described herein to the subject.

In some embodiments, an antibody or antigen-binding fragment thereof herein binds specifically to a free drug of an ADC. In some embodiments, the ADC comprises an auristatin. In some embodiments, the auristatin is monomethyl auristatin E (MMAE), monomethyl auristatin F (MMAF), or monomethyl auristatin D (MMAD). In some embodiments, the ADC is brentuximab vedotin, enfortumab vedotin, polatuzumab vedotin, disitamab vedotin, tisotumab vedotin, sigvotatug vedotin, belantamab mafodotin, or vorsetuzumab mafodotin,

In some embodiments, off-target effects of a free drug of an ADC in a subject is reduced by at least about 10%, e.g., by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or greater after administration of an antibody or antigen-binding fragment thereof described herein.

In some embodiments, a subject is a mammal. In some embodiments, a subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, a subject is a primate. In some embodiments, a subject is a human.

In some embodiments, a subject has, or is suspected of having, a disease, disorder, or condition. In some embodiments, a subject is at risk of developing a cancer. In some embodiments, a subject has been diagnosed with a cancer. In some embodiments, a subject has a solid tumor. In some embodiments, a subject has a hematological cancer.

A cancer can include, but is not limited to, a solid tumor, a hematological cancer (e.g., leukemia, lymphoma, or myeloma, e.g., multiple myeloma), a metastatic lesion, or a combination thereof. Examples of solid tumors include, but are not limited to, malignancies e.g., sarcomas and carcinomas, e.g., adenocarcinomas of the various organ systems, such as those affecting lungs, breast, ovaries, lymph nodes, genitals, gastrointestinal (e.g., colon) and genitourinary tracts (e.g., renal, urothelial, bladder cells, or prostate), pharynx, CNS (e.g., brain, neural, or glial cells), head and neck, skin (e.g., melanoma, e.g., a cutaneous melanoma), pancreas, and bones (e.g., a chordoma).

In some embodiments, a cancer comprises or is a lung cancer (e.g., a non-small cell lung cancer (NSCLC (e.g., a non-small cell lung cancer (NSCLC) with squamous and/or non-squamous histology or a NSCLC adenocarcinoma) or a small cell lung cancer (SCLC)), a skin cancer (e.g., a Merkel cell carcinoma or a melanoma (e.g., an advanced melanoma)), an ovarian cancer, a mesothelioma, a bladder cancer (e.g., a muscle-invasive bladder cancer (MIBC), a urothelial carcinoma), a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer (TNBC)), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hodgkin lymphoma), a myeloma (e.g., a multiple myeloma), a leukemia (e.g., a myeloid leukemia or a lymphoid leukemia), or a combination thereof.

In some embodiments, a cancer comprises or is a brain tumor, e.g., a glioblastoma, a gliosarcoma, or a recurrent brain tumor. In some embodiments, a cancer comprises or is a pancreatic cancer, e.g., an advanced pancreatic cancer. In some embodiments, a cancer comprises or is a skin cancer, e.g., a melanoma (e.g., a stage II-IV melanoma, an HLA-A2 positive melanoma, an unresectable melanoma, or a metastatic melanoma) or a Merkel cell carcinoma. In some embodiments, a cancer comprises or is a renal cancer, e.g., a renal cell carcinoma (RCC; e.g., a metastatic renal cell carcinoma). In some embodiments, a cancer comprises or is a breast cancer, e.g., a metastatic breast carcinoma or a stage IV breast carcinoma, e.g., a TNBC. In some embodiments, a cancer comprises or is a virus-associated cancer. In some embodiments, a cancer comprises or is an anal canal cancer (e.g., a squamous cell carcinoma of an anal canal). In some embodiments, a cancer comprises or is a cervical cancer (e.g., a squamous cell carcinoma of the cervix). In some embodiments, a cancer comprises or is a gastric cancer (e.g., an Epstein Barr Virus (EBV) positive gastric cancer or a gastric or gastro-esophageal junction carcinoma). In some embodiments, a cancer comprises or is a head and neck cancer (e.g., an HPV-positive or negative squamous cell cancer of the head and neck (SCCHN)). In some embodiments, a cancer comprises or is a nasopharyngeal cancer (NPC). In some embodiments, a cancer comprises or is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer comprises or is a hematological cancer. In some embodiments, a cancer comprises or is a leukemia, e.g., acute myeloid leukemia, chronic myeloid leukemia, acute lymphoblastic leukemia, chronic lymphocytic leukemia, chronic leukemia, or acute leukemia. In some embodiments, a cancer comprises or is a lymphoma, e.g., Hodgkin lymphoma (HL), non-Hodgkin's lymphoma, lymphocytic lymphoma, or DLBCL (e.g., a relapsed or refractory HL or DLBCL). In some embodiments, a cancer comprises or is a myeloma, e.g., multiple myeloma.

Administration of an antibody or antigen-binding fragment thereof described herein may be carried out in any convenient manner (e.g., injection, ingestion, transfusion, inhalation, implantation, or transplantation). In some embodiments, an antibody or antigen-binding fragment thereof described herein is administered by injection or infusion. In some embodiments, an antibody or antigen-binding fragment thereof described herein is administered transarterially, subcutaneously, intravenously, intradermally, intratumorally, intranodally, intramedullary, intramuscularly, or intraperitoneally. In some embodiments, an antibody or antigen-binding fragment thereof described herein is administered parenterally (e.g., intravenously, subcutaneously, intraperitoneally, or intramuscularly). In some embodiments, an antibody or antigen-binding fragment thereof described herein described herein is administered intravenously, intramuscularly, subcutaneously, or intraperitoneally by infusion or injection. In some embodiments, an antibody or antigen-binding fragment thereof described herein is administered intramuscularly. In some embodiments, an antibody or antigen-binding fragment thereof described herein is administered intravenously. In some embodiments, an antibody or antigen-binding fragment thereof described herein is administered subcutaneously. In some embodiments, an antibody or antigen-binding fragment thereof described herein is administered by intramuscular injection. In some embodiments, an antibody or antigen-binding fragment thereof described herein is administered by intravenous injection. In some embodiments, an antibody or antigen-binding fragment thereof described herein is administered by subcutaneous injection.

### Pharmaceutical Compositions

The present disclosure, among other things, provides pharmaceutical compositions comprising antibodies or antigen-binding fragments thereof that bind specifically to a free drug of an antibody drug conjugate (ADC) described herein in combination with one or more physiologically acceptable carriers, diluents, or excipients. When “a therapeutically effective amount” is indicated, a precise amount of a pharmaceutical composition comprising an antibody or antigen-binding fragment thereof described herein can be determined by a physician with consideration of individual differences in age, weight, immune response, and condition of a subject.

Pharmaceutical compositions described herein can comprise buffers including, but not limited to, neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of at least one contaminant (e.g., an endotoxin).

Pharmaceutical compositions described herein may be administered in any manner appropriate to a disease, disorder, or condition to be treated or prevented. Quantity and frequency of administration will be determined by such factors as condition of a subject, and type and severity of a subject's disease, disorder, or condition, although appropriate dosages may be determined by clinical trials.

Pharmaceutical compositions described herein may be in a variety of forms. These include, for example, liquid, semi-solid, and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for subcutaneous, intravenous, intramuscular, or intrasternal injection or infusion. In some embodiments, a pharmaceutical composition described herein is formulated for subcutaneous or intravenous injection of infusion. Pharmaceutical compositions described herein can be formulated for administered by using infusion techniques that are commonly known in immunotherapy (see, e.g., Rosenberg et al., 1988, New Eng. J. of Med. 319:1676, which is hereby incorporated by reference in its entirety).

### Kits

Provided herein, among other things, are kits comprising at least one antibody or antigen-binding fragment thereof that binds specifically to a free drug (e.g., MMAE) of an antibody drug conjugate (ADC) described herein, and instructions for use and/or administration. In some embodiments, a kit comprises least one antibody or antigen-binding fragment thereof described herein and a pharmaceutically acceptable carrier, and instructions for use and/or administration.

Also provided are kits for use in various methods described herein. In some embodiments, a kit comprises instructions for use in any method described herein. Instructions can comprise a description of administration of a first and second pharmaceutical composition to a subject to achieve an intended activity in a subject. A kit may further comprise a description of selecting a human suitable for treatment based on identifying whether the human is in need of the treatment. In some embodiments, instructions comprise a description of administering at least one antibody or antigen-binding fragment thereof described herein to a subject.

Instructions relating to administering a dose comprising at least one antibody or antigen-binding fragment thereof described herein generally include information as to dosage, dosing schedule, and route of administration for the intended treatment. Containers may be unit doses, bulk packages (e.g., multi-dose packages), or sub-unit doses. Instructions supplied in kits as described herein are typically written instructions on a label or package insert. A label or package insert may indicate that pharmaceutical compositions described herein are used for treating or preventing off-target effects of a free drug of an ADC in a subject.

Kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, the container may be an intravenous solution bag or a vial having a stopper pierce able by a hypodermic injection needle). A container may also have a sterile access port.

Kits may provide additional components, such as buffers and interpretive information. Normally, a kit can include a container and a label or package insert(s) on or associated with the container. In some embodiments, provided herein are articles of manufacture comprising contents of kits described herein.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which various aspects of innovations described herein belong. Although methods and materials similar or equivalent to those described herein can be used in the practice or testing of various aspects of innovations described herein, suitable methods and materials are described herein.

Various aspects of innovations described herein is further illustrated by the following example. An example is provided for illustrative purposes only. It is not to be construed as limiting the scope or content of various aspects of innovations described herein in any way.

### EXAMPLES

The following examples are provided so as to describe to the skilled artisan how to make and use methods and compositions described herein and is not intended to limit the scope of various aspects of innovations described herein.

### Example 1: Identification of Exemplary Payload Neutralizer-Binding Antibodies

The present Example demonstrates identification and generation of a series of exemplary payload neutralizing antibodies designed to bind free MMAE, but not MMAE conjugated to an antibody using a proprietary AI-based platform.

### Methods

Serially diluted exemplary payload neutralizing antibody variants were transferred to wells of plates containing immobilized MMAF-PEG-2-Biotin-Neutravidin (Anthem Biosciences) and incubated for 2 hours at room temperature. Plates were washed 3 times with TBS-T buffer, then DELFIA Eu-N1 anti-human IgG antibody was added and incubated for 30 minutes at room temperature. Plates were washed 5 times with TBS-T buffer, then a DELFIA Enhancement solution was added and incubated for 10 minutes. Time-resolved fluorescence was measured at 615 nm on a plate reader.

### Results

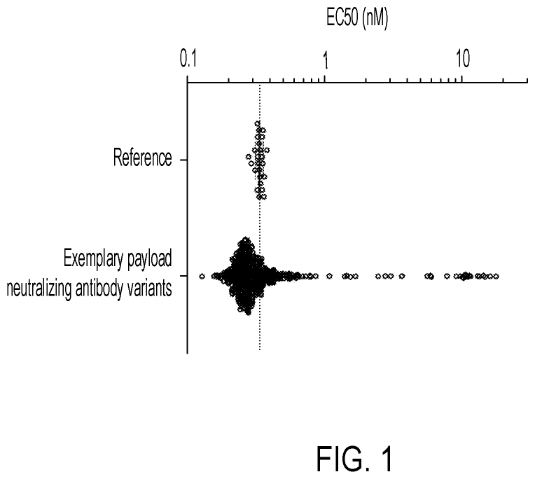

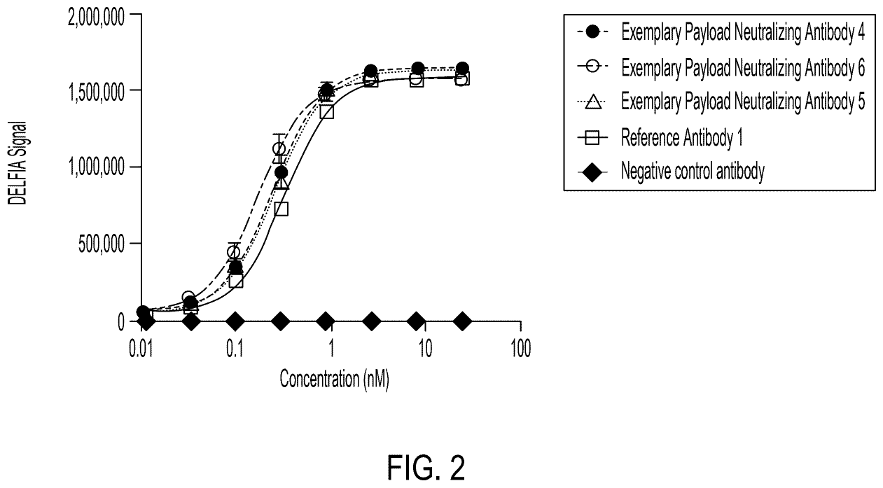

Exemplary payload neutralizing antibody variants showed improvement in binding compared to a reference payload neutralizing antibody (Reference) (FIG. 1) and exhibited binding to immobilized MMAF in a dose-dependent manner (FIG. 2). Exemplary payload neutralizing antibody variants that showed comparable or lower EC50 values compared to a reference antibody were selected to undergo a MMAE neutralization cytotoxicity assay.

### Example 2: In Vitro Characterization of Exemplary Payload Neutralizing Antibodies

The present Example demonstrates improved neutralization and binding affinity to free MMAE of exemplary payload neutralizing antibodies relative to payload neutralizing antibodies known in the art.

### Methods

Specific Neutralization of MMAE Cytotoxicity without Impairment of Antibody Drug Conjugated (ADC) Activity

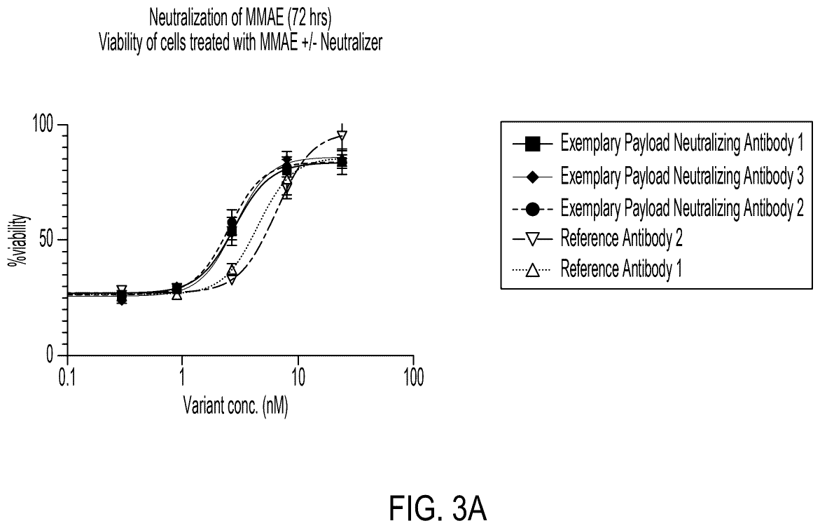

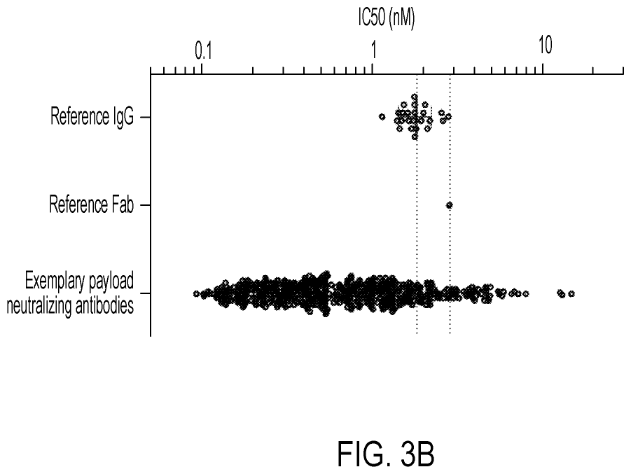

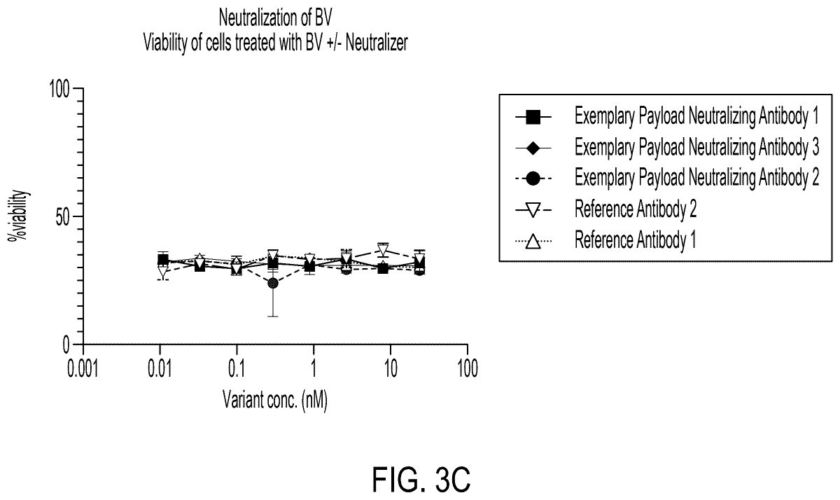

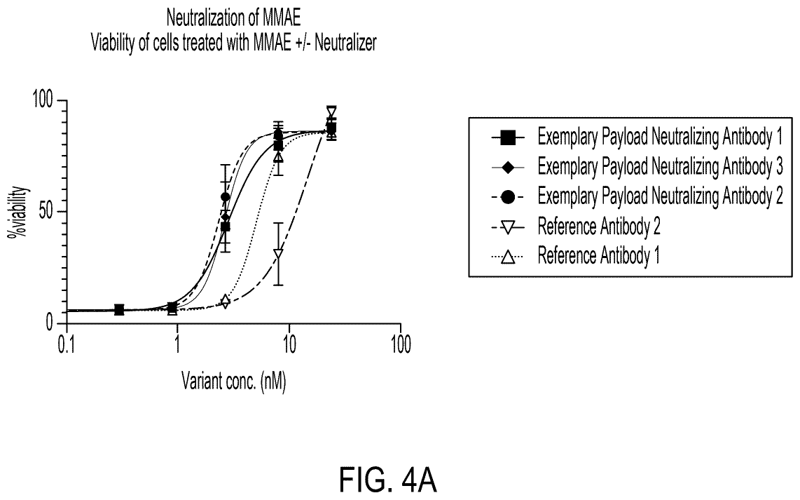

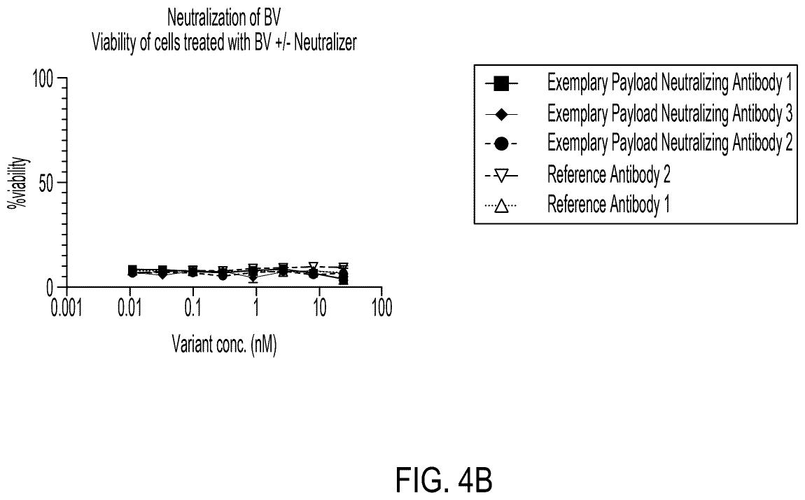

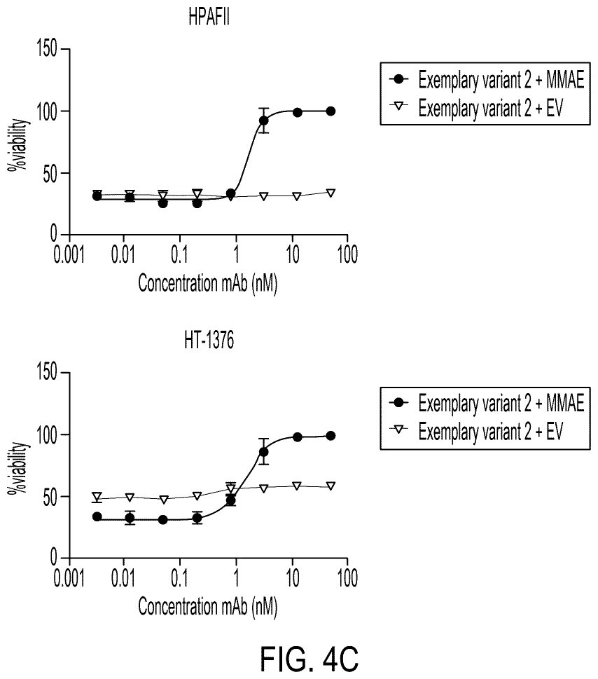

Kapras-299 cells were grown in RPMI (Gibco)+10% FBS in log phase, then seeded at a density of 2500 cells per well in 384-well flat bottom plates (Corning). Exemplary payload neutralizing antibody variants were serially diluted in PBS to achieve a final concentration between 0.01 nM to 28 nM. Media containing free MMAE at final concentration of 0.5 nM or Brentuximab Vedotin (BV) at final concentration of 0.08 nM were added to neutralizing antibody dilutions and incubated at room temperature for 30 minutes before added to individual wells. Cells were incubated for 3 days (FIG. 3) or 5 days (FIG. 4) at 37° C. in a humidified incubator with 5% CO2. After incubation, cell viability was assessed using cell titer glo one solution (Promega) following the manufacturer's protocol. A luminescence was recorded using a standard plate reader. Cell viability was determined as percentage of luminescence for treated cells divided by untreated cells. Observed cell viability was fitted to a nonlinear regression model using absolute IC50 method in GraphPad Prism 10.

In further experiments, HPAFII and HT-1376 cells were seeded in 384-well cell culture plates at a density of 1,250 and 1,000 cells per well, respectively, in 40 μL of cell culture medium and stored at 37° C., 5% CO₂ incubator (“Cell plate”) overnight. Exemplary Payload Neutralizing Antibody 2 lacking LALAPA Fc mutations was serially diluted 1:4 in PBS to achieve final concentrations ranging from 0.0031 nM to 50 nM (“Dilution plate”). Media containing free MMAE at a final concentration of 2 nM or Enfortumab Vedotin (EV) at a final concentration of 100 nM were prepared in 96-well plates (“Drug plate”), mixed with the Exemplary Payload Neutralizing Antibody 2 dilutions prepared above, incubated at room temperature for 30 minutes, and then added to the HPAFII and HT-1376 cell plates. Cells were incubated for 5 days at 37° C. in a humidified incubator with 5% CO2. After incubation, cell viability was assessed using CellTiter-glo (Promega) following the manufacturer's protocol. Luminescence was recorded using a standard plate reader (Envision, PerkinElmer). Percent cell viability was calculated from luminescence observed with cells treated with MMAE/EV and antibody over luminescence observed with cells in the absence of MMAE/EV treatment. Cell viability was fitted to a nonlinear regression model using the absolute IC50 method in GraphPad Prism 10.

### Kinetics of Biotinylated Exemplary Payload Neutralizing Antibody Fragment Variants Binding to MMAE Using Surface Plasmon Resonance (SPR) at 37° C.

To evaluate kinetics of exemplary payload neutralizing antibody fragments binding to MMAE, a single-cycle capture kinetics approach was implemented using a Biacore 8K+ (Cytiva). A Series S Biotin CAPture kit was used according to the instructions provided by the manufacturer. Each biotinylated exemplary payload neutralizing antibody fragment was prepared at 50 μg/mL and captured for 30 seconds at a flow rate of 10 μL min⁻¹ to achieve a capture level of approximately 2000 RU. MMAE was prepared in HBS-EP⁺, pH 7.6 (10 mM HEPES pH 7.4, 150 mM NaCl, 3 mM EDTA and 0.05% v/v surfactant P20) running buffer and injected over the captured antibody surface at 8 concentrations in a 2-fold serial dilution series from 0.4 nM to 50 nM. Each MMAE concentration was injected sequentially from low concentration to high concentration for 180 seconds at a flow rate of 30 μL min⁻¹, and the complex was allowed to dissociate for 3600 seconds. The assay was performed at 37° C. with technical replicates at a collection rate of 1 Hz. Kinetic parameters for the concentration series were obtained by double referencing and globally fitting data to a 1:1 binding model using the Biacore Insight Evaluation software. It was recommended to attain at least a 5% decrease in binding response for the highest analyte concentration tested during a dissociation phase to accurately measure slow dissociation rate constants. For binding interactions reaching this recommendation during the 3600 seconds dissociation phase, kinetic and affinity values were reported, and results were expressed as average±standard deviation of one independent experiment with technical replicates. For binding interactions that did not satisfy this condition, dissociation rate constant ka was fixed at 1.42E-05 and an equilibrium dissociation rate constant K_{D} was estimated and reported as an apparent affinity.

### Non-Specific Binding of Exemplary Payload Neutralizing Antibodies to Human Insulin and Cytosolic CHO Extract

DELFIA, a time-resolved fluorescence (TRF) intensity technology, was used to determine binding of test articles and control antibodies to the nonspecific targets cytosolic CHO extract (AGN-000045-007 Generate Biomedicines production, prepared using ExpiCHO cells via Mem-PER™ Plus Membrane Protein Extraction Kit (Thermo 89842)) and insulin (Sigma Aldrich). This assay was adapted from Tiller et al., 2008, J. Immunol. Methods. 329 (1-2): 112-24, the contents of which are incorporated by reference in its entirety.

Next, 384 well Maxisorp plates were incubated with 25 μL human insulin or cytosolic CHO extract (10 μg/mL and 5 μg/mL respectively) overnight at 4° C. and washed with TBS, then blocked with 5% BSA in TBST blocking buffer for 30 minutes at 37° C. After washing, the plates were incubated with test articles and TBS with no antibody (background control) for one hour at 37° C. Following this, plates were washed with TBS and incubated with Eu-labeled anti-human IgG secondary antibody (0.1 μg/mL) for 30 minutes at 37° C., followed by an additional wash and incubation with DELFIA enhancement solution for 10 minutes following shaking of the plate for 5 minutes. TRF for each well was detected using the excitation wavelength of 330 nm and emission wavelength of 620 nm with delay of 400 μs for a duration of 300 ms.

The “PSR Score” for each antigen was calculated as follows: the average fluorescence signal (FL) of technical replicates was divided by the average background FL signal.

$$PSR Score = \frac{F l u o r e s cence Signal of Sample}{Background Fluorescence Signal}$$

The binding control antibodies used were certolizumab and bococizumab (Generate Biomedicines). Bococizumab is a humanized and affinity matured anti-PCSK9 antibody that was shown to exhibit a short PK, poor biodistribution after sub-cutaneous dosing and evoke high levels of anti-drug antibodies in patients (see Cunningham et al., 2021, mAbs. 13 (1): 1999195; Ridker et al., 2017, New Eng. J. of Med. 376 (16): 1517-1526, each of which is incorporated by reference herein in its entirety). Additionally, bococizumab exhibits high retention times in cross interaction chromatography which has been shown to correlate with higher clearance rates in mice (see Jain et al., 2017, PNAS. 114 (5): 944-949; Kelly et al., 2015, mAbs. 7 (4): 770-7, each of which is incorporated by reference in its entirety). Certolizumab is a clinical antibody exhibiting favorable developability properties, including low non-specific binding.

### Competitive DELFIA Binding Assay to Assess Specificity of Exemplary Payload Neutralizer Variants for Free MMAE

Exemplary payload neutralizing antibody variants were characterized using a competitive DELFIA binding assay with increasing concentrations of free MMAE and Brentuximab Vedotin (ADC).

Exemplary payload neutralizing antibody variants were diluted to 1 nM and incubated with either MMAE or ADC at concentrations between 0.002 to 100 nM for 30 minutes at room temperature. Solutions were added to wells of plates containing immobilized MMAF-PEG-2-Biotin-Neutravidin and incubated for 2 hours at room temperature. Plates were washed 3 times with TBS-T buffer, then DELFIA Eu-N1 anti-human IgG antibody was added and incubated for 30 minutes at room temperature. Plates were washed 5 times with TBS-T buffer, added DELFIA Enhancement solution, and incubated for 10 minutes. Time-resolved fluorescence was read at 615 nm on a plate reader.

### AlphaLISA Homogeneous Binding Assay for Exemplary Payload Neutralizing Antibody and Payload

Binding of exemplary payload neutralizing antibody variants to payload (biotinylated MMAF) was characterized using an AlphaLISA assay with increasing concentrations of neutralizing antibody variants and 0.1 nM MMAF-PEG2-Biotin.

A 4× dilution series of exemplary payload neutralizing antibody variants at 96-0.004 nM (final concentrations: 24-0.001 nM) was incubated with 0.4 nM MMAF-PEG-2-Biotin (final concentration: 0.1 nM) for 1 hour at room temperature. Following the incubation, 4× concentrations of AlphaLISA Anti-Human IgG (Fc specific) Acceptor Beads from Revvity (Cat #: AL103) were added to the reaction and incubated for 1 hour at room temperature. Subsequently, 4× concentrations of AlphaScreen Streptavidin Donor Beads from Revvity (Cat #: 6760002) were added and incubated for an additional hour at room temperature. The final bead concentrations of acceptor and donor beads were 20 μg/ml for both. AlphaLISA signals were measured by a plate reader. Results are shown as the mean of n=3 technical replicates, fitted using a dose-response stimulation curve with a bell-shaped model in GraphPad Prism 10 software.

### Kinetics of Biotinylated Exemplary Payload Neutralizing Antibody Fragment Variants Binding to MMAE Using Kinetic Exclusion Assay (KinExA)

KinExA was used to measure the affinity and kinetic parameters for binding between Exemplary Payload Neutralizing Antibodies lacking LALAPA Fc mutations and MMAE. In this assay, the interaction occurs in solution, and free antibody is captured on a solid phase coated with biotinylated MMAF. MMAF shares a nearly identical structure with MMAE and differs only at the carboxyl group near the C-terminus. This carboxyl group is also the site of biotinylation.

To determine affinity, a dual-curve approach was used in which MMAE was the titrated interaction partner in the background of a constant concentration of Exemplary Payload Neutralizing Antibodies lacking LALPA Fc mutations. Both Payload Neutralizing Antibodies and MMAE were prepared using 1× PBS with 1 mg/ml BSA and 0.02% sodium azide and allowed to reach equilibrium before running the sample through a solid phase containing capture antigen, to determine percent free Exemplary Payload Neutralizing Antibody in solution. Percent of captured free Exemplary Payload Neutralizing Antibodies in solution was detected by a fluorescent secondary antibody and was directly proportional to the signal generated by the KinExA 4000. Parameters for affinity conditions for each of the tested Payload Neutralizing Antibodies are shown in Table 2. After equilibration, in separate experiments, concentration-controlled and KD-controlled sample sets were injected into a column containing the solid phase of streptavidin glass beads coated with 30 μg/ml biotinylated MMAF with a sampling time indicated in Table 2, at a flow rate of 0.25 mL/min. The percent of free Payload Neutralizing Antibodies was detected using an Alexa-Fluor-647 Anti-human IgG (H+L) antibody at 0.5 μg/mL injected for 120 s at 0.25 mL/min. Each curve was an independent experiment with technical replicates. The KinExA Pro software performed the nonlinear least squares regression analysis of the measured data to determine the best-fit values of the K_{D} and active concentration using N-curve analysis of the K_{D}-controlled and concentration-controlled curves, respectively, which fits the data to a 1:1 reversible bimolecular interaction using MMAE as the concentration reference. The software reports the affinity parameters including the 95% confidence intervals and fit quality metrics.

For association rate constant ka (M-1s-1) determination, measurements were collected using a Kinetics Direct method, where pre-equilibrium binding signals were acquired as a function of time. Parameters for kinetics measurements for each of the tested Exemplary Payload Neutralizing Antibodies are shown in Table 2. The mixture was repeatedly sampled with timing indicated in Table 2 at a flow rate of 0.25 mL/min. Each sampling measured the percent free Payload Neutralizing Antibody captured by streptavidin beads coated with 30 μg/ml biotinylated MMAF using an Alexa-Fluor-647 Anti-human IgG (H+L) antibody at 0.5 μg/mL injected for 120 s at 0.25 mL/min. Using a nonlinear regression to a 1:1 binding model, kinetic parameters were extracted from the time-course signals using the KD and active concentrations determined in the n-curve analyses. The software reports the kinetic parameters, the 95% confidence intervals, and fit quality metrics.

TABLE 2
KinExA Parameters

| | Kinetics Conditions | Affinity Conditions (Sampling |
|---|---|---|
| CBP (IgG) | (Sampling time) | time) |
| Reference Payload | 50 pM CBP | 100 pM CBP; 10 nM titrant (108 s) |
| Neutralizing Antibody 1 | 2 nM titrant (144 s) | 5 nM CBP; 100 nM titrant (3 s) |
| | | 15 nM CBP; 200 nM titrant (1 s) |
| Reference Payload | 50 pM CBP | 100 pM CBP; 10 nM titrant (108 s) |
| Neutralizing Antibody 2 | 1 nM titrant (144 s) | 2 nM CBP; 100 nM titrant (6 s) |
| Exemplary Payload | 100 pM CBP | 10 pM CBP; 500 pM titrant (840 s) |
| Neutralizing Antibody 1 | 500 pM titrant (84 s) | 200 pM CBP; 5 nM titrant (36 s) |
| Exemplary Payload | 100 pM CBP | 10 pM CBP; 500 pM titrant (960 s) |
| Neutralizing Antibody 2 | 2 nM titrant (84 s) | 200 pM CBP; 5 nM titrant (72 s) |
| Exemplary Payload | 100 pM CBP | 10 pM CBP; 500 pM titrant (960 s) |
| Neutralizing Antibody 3 | 2 nM titrant (84 s) | 200 pM CBP; 5 nM titrant (108 s) |

### Binding of Exemplary Payload Neutralizing Antibody Fragment Variants to Fc Gamma Receptors Using SPR

To evaluate the binding of Exemplary Payload Neutralizing Antibody 2 to a panel of human Fc gamma receptors, SPR measurements were performed using a Biacore 8K+ at 25° C. Biotinylated recombinant FcγRI, FcγRIIa-R167, FcγRIIa-H167, FcγRIIb, FcγRIIIa-F176, FcγRIIIa-V176, FcγRIIIb-NA1, and FcγRIIIb-NA2 were prepared at 0.3 μg/mL and captured for 60 s at a flow rate of 10 μL min−1 to achieve a capture level of approximately 75 RU. Exemplary Payload Neutralizing Antibody 2, isotype-hIgG1 and isotype-hIgG1-LALAPA were prepared in 1× HBS-EP+ and injected over the captured Fc gamma receptor surface at 5,000 nM. Each analyte was injected in a single cycle at a flow rate of 10 μL min−1 for 180 s, and the complex was allowed to dissociate for 600 s using 1× HBS-EP+ as the running buffer. The surface was regenerated by injecting one pulse of a regeneration solution prepared from 3 parts 8 M guanidine hydrochloride and 1 part 1 M sodium hydroxide for 120 s at a flow rate of 10 μL min−1 in between each cycle. The assay was performed at 25° C. with technical replicates. Fc gamma receptor capture level and antibody binding response report points were obtained using the Biacore Insight Evaluation software after double referencing. Antibody binding responses were normalized to the human Fc gamma receptor capture levels. The response for a buffer injection was 5 RU, while the average capture level for all receptors tested was 75 RU. A normalized response≤0.06 (equivalent to 5 RU at 75 RU capture) was classified as non-binding; >0.06 was considered binding.

### Binding of Exemplary Payload Neutralizing Antibody Fragment Variants Binding to Human C1q Using BLI

To evaluate the binding of Exemplary Payload Neutralizing Antibody 2 to human C1q, Biolayer Interferometry (BLI) measurements were performed. All samples were diluted to their final concentration in 1× kinetics buffer. Exemplary Payload Neutralizing Antibody 2 and isotype controls isotype-hlgG1 and isotype-hIgG1-LALAPA were captured using anti-human Fab biosensors in 16-channel mode. All biosensors were hydrated for at least 10 min in 1× kinetics buffer before use. Measurements were performed in 384 tilted well microplates with at least 85 μL at a 4 mm sensor offset. The plate temperature was set to 30° C. and shaker speed to 1000 RPM for all steps. Sensors were placed into wells containing 1× kinetics buffer for 60 s to establish a baseline prior to antibody capture. To capture the panel of antibodies on the anti-human Fab sensor surface, sensors were placed in wells containing 20 μg/mL of Exemplary Payload Neutralizing Antibody 2, and isotype controls isotype-hIgG1 and isotype-hlgG1-LALAPA for 180 s to achieve a minimum loading response of 0.8 nm. A second baseline was recorded by placing sensors into wells containing 1× kinetic buffer prior to the association phase. Sensors were placed into wells containing a 7-point, 2-fold serial dilution from 250 nM to 3.9 nM of human C1q for 300 s before dissociating in 1× kinetics buffer for 300 s. A buffer well was used as a reference sample and was subtracted from the data to correct for bulk effects. Replicate measurements were performed in a single experiment. The raw data was aligned to the y-axis by aligning the data at the end of the baseline step, inter-step correction was used to align the start of the dissociation to the end of the association phase and Savitzky-Golay filtering was applied. Report points of the capture level and binding response of human C1q at 250 nM were obtained. With this technique, a response of ≤0.1 nm is typically considered to be a non-binding interaction, while a response >0.1 nm is considered to be a binding interaction.

### Results

Specific Neutralization of Monomethyl Auristatin E (MMAE) Cytotoxicity without Impairment of Antibody Drug Conjugated (ADC) Activity

As shown in FIG. 3A, addition of payload neutralizing antibodies to media containing free MMAE reduced the cytotoxic effects of MMAE in a dose-dependent manner. In this experiment, viability of cells incubated with 0.5 nM MMAE with payload neutralizing antibodies was assessed after 72 hours. In this same experiment, cytotoxicity of Brentuximab Vedotin was not interfered with by addition of exemplary payload neutralizing antibodies, indicating that exemplary payload neutralizing antibodies do not interact with intact ADC (FIG. 3C). Exemplary payload neutralizing antibodies had IC50 values of 0.096-14.6 nM at 72 hours suggesting that free MMAE was rendered inactive for the duration of the assay (Table 3; FIG. 3B).

TABLE 3
MMAE neutralization IC50

| | Potency IC₅₀ (nM) | Potency IC₅₀ (nM) |
|---|---|---|
| Protein | In 72-hour assay | in 120-hour assay |
| Reference | 0.67 | 14.26 |
| Affinity-matured reference | 0.22 | 5.0 |
| Exemplary Payload Neutralizing | 0.16 | 2.7 |
| Antibody 1 | | |
| Exemplary Payload Neutralizing | 0.15 | 2.5 |
| Antibody 2 | | |
| Exemplary Payload Neutralizing | 0.14 | 2.5 |
| Antibody 3 | | |

As shown in FIG. 4A and Table 3, addition of Exemplary Payload Neutralizing Antibodies 1, 2, or 3 to media containing free MMAE reduced cytotoxic effects of MMAE in a dose-dependent manner. In this experiment, viability of cells incubated with 0.08 nM Brentuximab Vedotin or 0.5 nM MMAE+Exemplary Payload Neutralizing Antibodies 1, 2, or 3 was assessed after 120 hours. Cytotoxicity of Brentuximab Vedotin was not interfered with by addition of Exemplary Payload Neutralizing Antibodies 1, 2, or 3 (FIG. 4B).

Similarly, as shown in FIG. 4C, the addition of Exemplary Payload Neutralizing Antibody 2 to media containing free MMAE reduced cytotoxicity of MMAE in a dose-dependent manner. In this experiment, viablity of cells incubated with 2 nM MMAE or 100 nM enfortumab vedotin+Exemplary Payload Neutralizing Antibody 2 was assessed after 120 hours. Cytotoxicity of enfortumab vedotin was not interfered with by addition of Exemplary Payload Neutralizing Antibody 2 (FIG. 4C)

### Kinetics of Biotinylated Exemplary Payload Neutralizing Antibody Fragment Variants Binding to Monomethyl Auristatin E (MMAE) Using SPR at 37° C.

Kinetics and affinity of biotinylated exemplary payload neutralizing antibody fragment variants binding to monomethyl auristatin E (MMAE) were obtained by SPR at 37° C. Exemplary Payload Neutralizing Antibody Fragments 1, 2, and 3 correspond to Fab fragments of Exemplary Payload Neutralizing Antibodies 1, 2, and 3, respectively. Parameters displayed in the table represent the following: ka refers to association rate constant, kd refers to dissociation rate constant, KD refers to equilibrium dissociation constant. Results are expressed as average±standard deviation of one independent experiment with technical replicates. Exemplary Payload Neutralizing Antibody Fragments 1, 2, and 3 exhibit high affinity for MMAE relative to Reference Antibody Fragments 1 and 2 (Table 4).

TABLE 4
Affinity for free MMAE at 37° C.

| Protein | kₐ (10⁵M⁻¹s⁻¹) | k_{d} (10⁻⁵ s⁻¹) | KD (10⁻¹²M) |
|---|---|---|---|
| Reference Antibody | 7.03 ± 1.61 | 18.79 ± 3.56 | 268.43 ± 10.96 |
| Fragment 1 | | | |
| Reference Antibody | 4.98 ± 0.83 | 2.80 ± 0.42 | 56.38 ± 0.92 |
| Fragment 2 | | | |
| Exemplary Payload | 8.36 ± 3.49 | 1.42 | <18.60 ± 7.77 |
| Neutralizing Antibody | | | |
| Fragment 1 | | | |
| Exemplary Payload | 7.68 ± 3.66 | 1.42 | <20.85 ± 9.93 |
| Neutralizing Antibody | | | |
| Fragment 2 | | | |
| Exemplary Payload | 4.01 ± 0.91 | 1.42 | <36.32 ± 8.26 |
| Neutralizing Antibody | | | |
| Fragment 3 | | | |

### Non-Specific Binding of Exemplary Payload Neutralizing Antibodies to Human Insulin and Cytosolic CHO Extract

All five exemplary payload neutralizing antibodies tested exhibited lower binding to both antigens compared to high binding control (Table 5). Exemplary Payload Neutralizing Antibody 2, showed least non-specific binding, followed by Exemplary Payload Neutralizing Antibody 1 and Exemplary Payload Neutralizing Antibody 3.

In summary, PSR scores represent the extent to which payload neutralizing antibodies and low and high binding control antibodies (certolizumab and bococizumab) bind to human insulin and cytosolic CHO extract (the higher the score, the higher binding is). All three exemplary payload neutralizing antibodies exhibited much lower non-specific binding compared to high binding controls for both antigens.

TABLE 5
Binding of exemplary payload neutralizing antibodies
to human insulin and cytosolic CHO extract.

| | Insulin | Cytosolic CHO |
|---|---|---|
| | Binding | Extract Binding |
| Name | PSR Score | PSR Score |
| Reference Antibody | 867.06 | 426.32 |
| 1 | | |
| Reference Payload | 834.8 | 497.79 |
| Neutralizing | | |
| Antibody 2 | | |
| Exemplary Payload | 72.39 | 60.22 |
| Neutralizing | | |
| Antibody 1 | | |
| Exemplary Payload | 31.38 | 25.91 |
| Neutralizing | | |
| Antibody 2 | | |
| Exemplary Payload | 1517.09 | 792.68 |
| Neutralizing | | |
| Antibody 3 | | |
| Low binding | 47.57 | 54.47 |
| control | | |
| (certolizumab) | | |
| High binding | 2182.46 | 1216.38 |
| control | | |
| (bococizumab) | | |

### Competitive DELFIA Binding Assay to Assess Specificity of Exemplary Payload Neutralizing Antibody Variants for Free MMAE

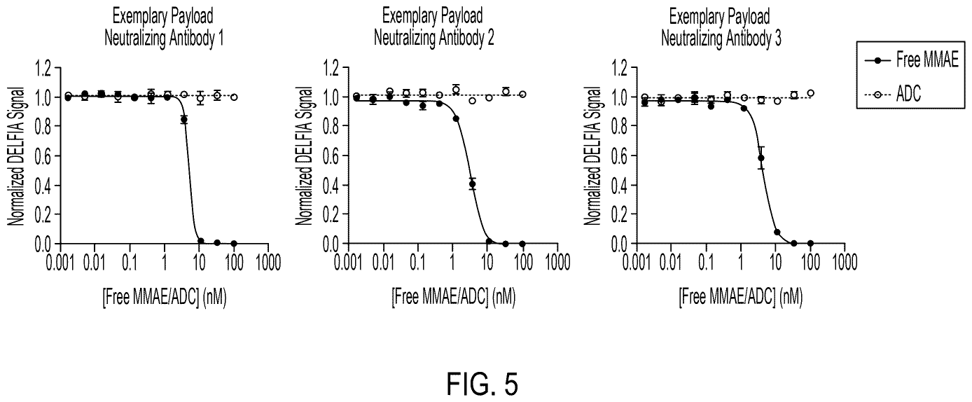

As shown in FIG. 5, binding of Exemplary Payload Neutralizing Antibodies 1, 2, and 3 to immobilized MMAF decreased with increasing concentrations of free MMAE with IC50 values of 5.0, 3.2, and 4.4 nM, respectively. In contrast, no decrease in binding signal was observed with Brentuximab Vedotin (ADC). This indicates that exemplary payload neutralizing antibodies disclosed herein selectively bound free MMAE.

### AlphaLISA Homogeneous Binding Assay for Exemplary Payload Neutralizing Antibodies and Payload

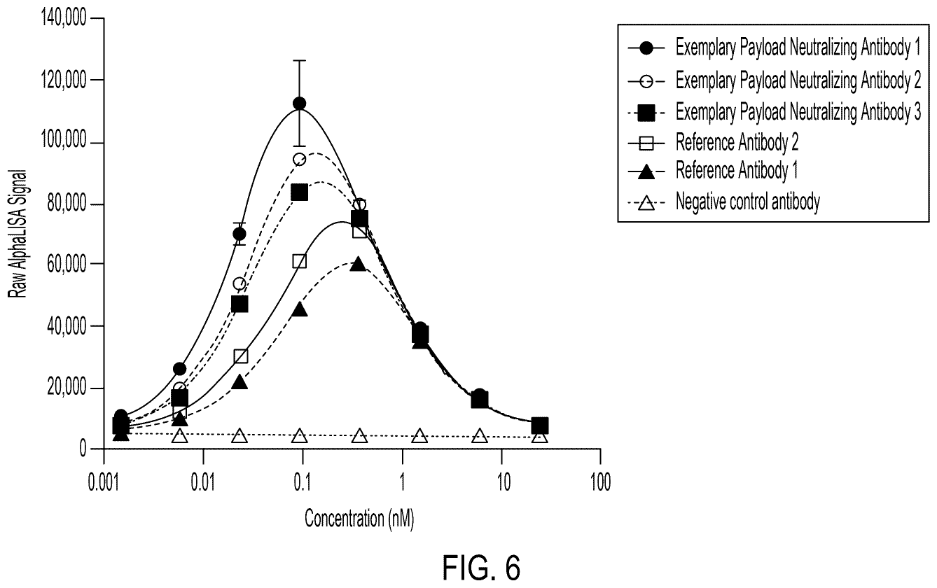

Binding of exemplary payload neutralizing antibody variants to payload (biotinylated MMAF) was measured using an AlphaLISA homogeneous binding assay. As shown in FIG. 6, AlphaLISA signals increased in a dose-dependent manner. Exemplary Payload Neutralizing Antibodies 1, 2, and 3 exhibited stronger binding to payload compared to Reference Antibodies 1 and 2, while human isotype antibody (Negative control antibody) exhibited background-level signals. Reduction in signals at higher concentrations is likely due to a hook effect, where binding sites on AlphaLISA beads become saturated, and excess exemplary payload neutralizing antibodies begin to inhibit signal. Importantly, binding signal peaks shifted to higher concentrations for reference antibodies exhibiting weaker binding signals, suggesting that these are low-affinity antibodies for payload compared to Exemplary Payload Neutralizing Antibodies 1, 2, and 3.

### Kinetics of Biotinylated Exemplary Payload Neutralizing Antibody Fragment Variants Binding to MMAE Using Kinetic Exclusion Assay (KinExA)

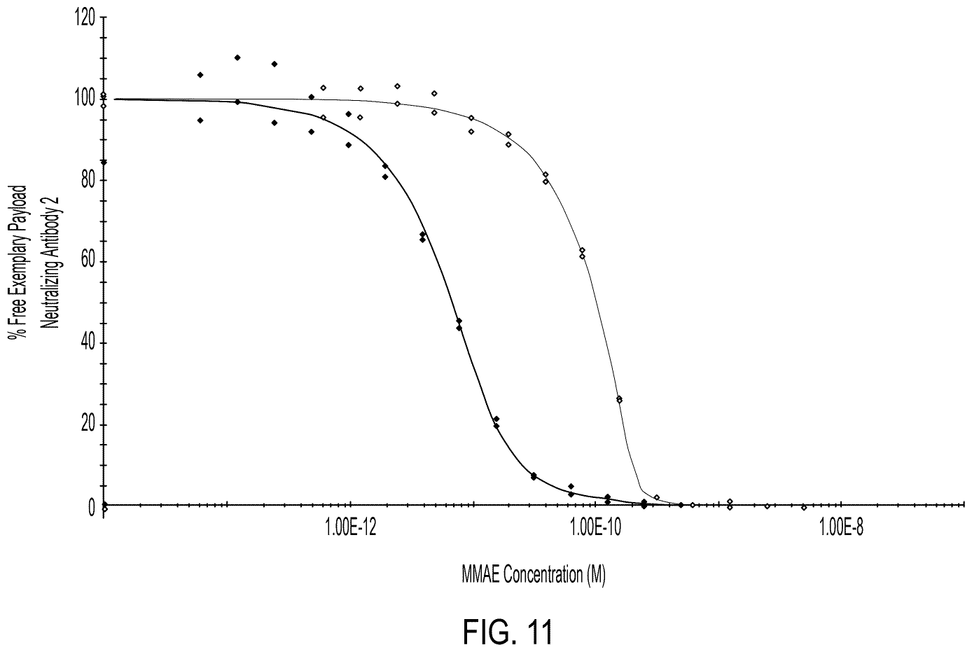

KinExA was employed to assess the affinity and kinetics of Exemplary Payload Neutralizing Antibodies lacking LALAPA Fc mutations binding to free MMAE. Affinity and Kinetics data is shown in Table 6, and an Equilibrium Binding Curve for Exemplary Payload Neutralizing Antibody 2 is shown in FIG. 11. The data demonstrate that Exemplary Payload Neutralizing Antibodies bind to MMAE with stronger affinities than Reference Payload Neutralizing Antibodies.

TABLE 6
Kinetics and Affinity Data for Exemplary Payload
Neutralizing Antibodies, measured by KinExA

| CBP | kₐ | k_{d} | K_{D} | % CBP |
|---|---|---|---|---|
| (IgG) | (10⁵M⁻¹s⁻¹) | (10⁻⁶ s⁻¹) | (10⁻¹²M) | Activity |
| Reference | 1.49 | 60.10 | 402.17 | 92.91 |
| Payload | (1.18-1.83) | | (270.54-588.57) | (73.45-114.75) |
| Neutralizing | | | | |
| Antibody 1 | | | | |
| Reference | 3.06 | 9.20 | 30.04 | 99.27 |
| Payload | (2.84-3.27) | | (19.52-43.18) | (85.82-113.98) |
| Neutralizing | | | | |
| Antibody 2 | | | | |
| Exemplary | 6.24 | 1.32 | 2.12 | 87.59 |
| Payload | (5.48-6.99) | | (1.02-3.64) | (71.97-103.33) |
| Neutralizing | | | | |
| Antibody 1 | | | | |
| Exemplary | 2.28 | 0.41 | 1.78 | 100.92 |
| Payload | (1.88-2.69) | | (0.63-3.51) | (80.39-124.27) |
| Neutralizing | | | | |
| Antibody 2 | | | | |
| Exemplary | 1.99 | 1.86 | 6.81 | 71.81 |
| Payload | (1.64-2.35) | | (4.70-9.43) | (55.34-90.00) |
| Neutralizing | | | | |
| Antibody 3 | | | | |

### Binding of Exemplary Payload Neutralizing Antibody Fragment Variants Binding to Fc Gamma Receptors Using SPR

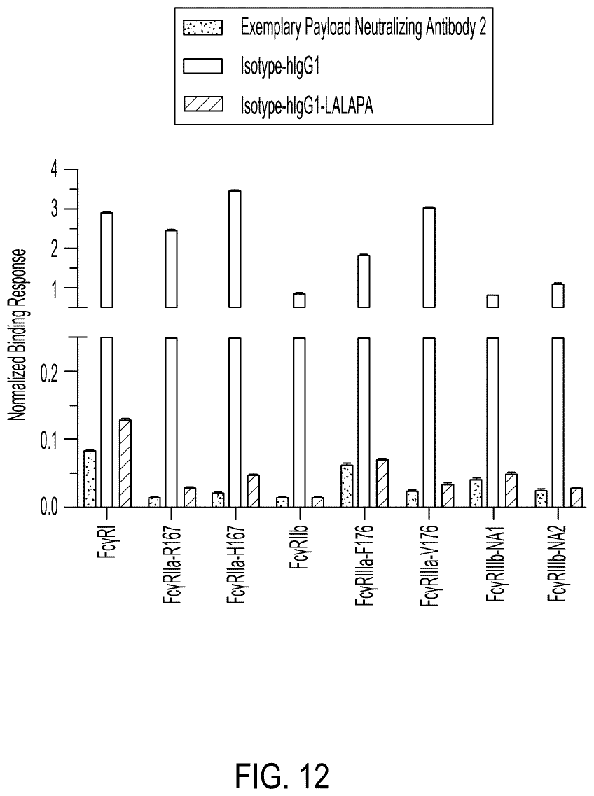

Binding of Exemplary Payload Neutralizing Antibody 2 to a panel of human Fc gamma receptors was evaluated using SPR. As shown in FIG. 12, Exemplary Payload Neutralizing Antibody 2 exhibited reduced binding to FcγRI and FcγRIIIa-F176, and no detectable binding to FcγRIIa-R167, FcγRIIa-H167, FcγRIIb, FcγRIIIa-V176, FcγRIIIb-NA1, and FcγRIIIb-NA2, relative to isotype-hIgG1, similar to the isotype-hlgG1-LALAPA control.

### Binding of Exemplary Payload Neutralizing Antibody Fragment Variants Binding to Human C1q Using BLI

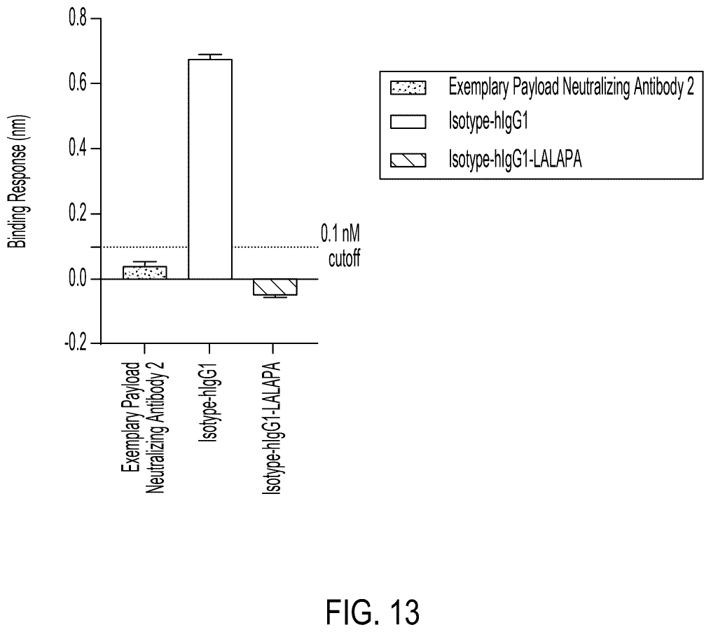

Binding of Exemplary Payload Neutralizing Antibody 2 to C1q was evaluated using BLI. As shown in FIG. 13, no binding of Exemplary Payload Neutralizing Antibody 2 to human C1q was detected.

### Example 3: Pharmacokinetics/Pharmacodynamics of Exemplary Payload Neutralizing Antibodies In Vivo

The present Example demonstrates pharmacokinetics and pharmacodynamics of exemplary payload neutralizing antibodies in vivo, which can be administered with ADCs to prevent and/or reduce free drug (e.g., MMAE) levels.

### Pharmacokinetics Study of Exemplary Payload Neutralizing Antibodies

### Methods

This study evaluated pharmacokinetics (PK) of five payload neutralizing antibodies: three payload neutralizing antibody variants (Exemplary Payload Neutralizing Antibodies 4, 5, and 6), a low potency control, and a reference antibody (Reference Antibody 1). Exemplary Payload Neutralizing Antibodies 4, 5, and 6 correspond to Exemplary Payload Neutralizing Antibodies 1, 2, and 3 lacking the L234A, L235A, and P329A (LALAPA) mutation, numbering according to EU index. Female SCID mice (6-8 weeks old) were randomized based on body weight into groups of four and received a single intravenous bolus injection of 10 mg/kg of a payload neutralizing antibody. Serum samples were collected at multiple time points: 1 hour, 4 hours, 24 hours, 3 days, and 7 days post-injection. Collected samples were analyzed using GyroLab to determine serum concentrations of each antibody.

### Results

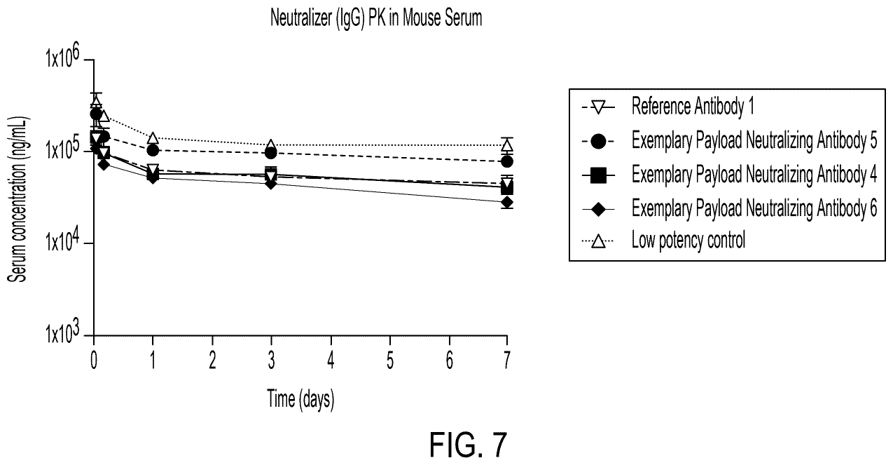

All five payload neutralizing antibodies exhibited similar PK profiles over the 7-day period (FIG. 7). Low potency control showed the most favorable PK properties, followed by Exemplary Payload Neutralizing Antibody 5. Exemplary Payload Neutralizing Antibody 4 and Reference Antibody 1 had comparable PK behavior.

Pharmacokinetics Study of Co-Administration of Brentuximab Vedotin with an Exemplary Payload Neutralizing Antibody

### Methods

This study evaluated the pharmacokinetics (PK) of co-administration of Brentuximab Vedotin (BV, 10 mg/kg) with Exemplary Payload Neutralizing Antibody 1 at doses of 3 mg/kg and 30 mg/kg, and a low potency control at 30 mg/kg. Female SCID mice (7-8 weeks old) were randomized based on body weight and injected intravenously with either BV alone or a combination of BV and a payload neutralizing antibody. Plasma-EDTA samples were collected at 24 hours, 7 days, and 28 days. Collected samples were analyzed using mass spectrometry to determine plasma concentrations of each antibody.

### Results

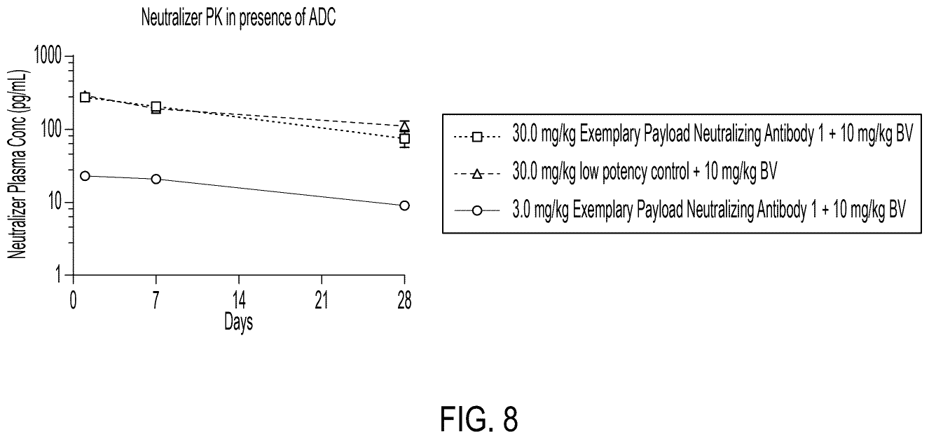

Co-administration of Brentuximab Vedotin with 3 mg/kg of Exemplary Payload Neutralizing Antibody 1 exhibited linear PK behavior over a 28-day period. Co-administration of Brentuximab Vedotin with either 30 mg/kg of Exemplary Payload Neutralizing Antibody lor 30 mg/kg of a low potency control had comparable linear PK behavior (FIG. 8).

### Pharmacokinetics/Pharmacodynamics Study to Characterize MMAE Engagement

### Methods

This study aimed to assess target engagement by measuring changes in free MMAE levels following co-administration of Brentuximab Vedotin (BV, 10 mg/kg) with Exemplary Payload Neutralizing Antibody 1 at doses of 3 mg/kg and 30 mg/kg, and a low potency control at 30 mg/kg. Female SCID mice (7-8 weeks old) were randomized based on body weight and injected intravenously with either BV alone or a combination of BV and a payload neutralizing antibody. Plasma-EDTA samples were collected at 15 minutes, 8 hours, 24 hours, 48 hours, 72 hours, and 7 days. Plasma samples were processed and analyzed via mass spectrometry to quantify free MMAE levels.

### Results

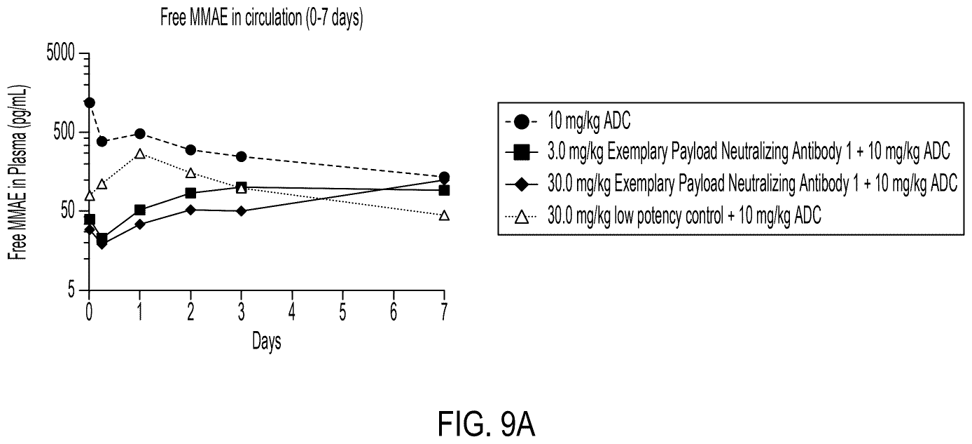

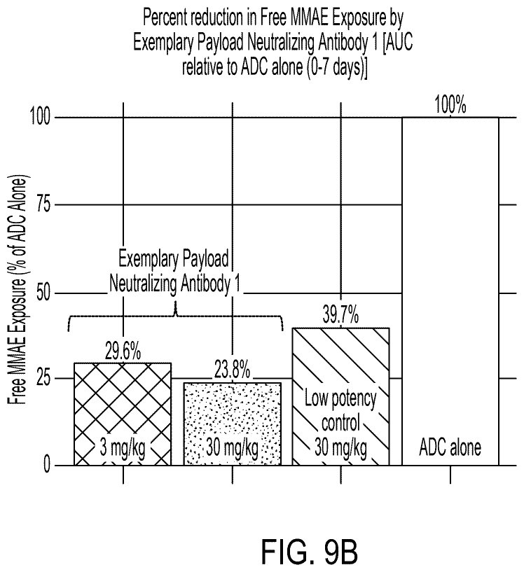

Co-administration of Brentuximab Vedotin (BV, 10 mg/kg) with Exemplary Payload Neutralizing Antibody 1 at doses of 3 mg/kg and 30 mg/kg resulted in a dose-dependent reduction in free MMAE plasma levels (FIG. 9A). Free plasma MMAE Cmax for BV alone was 1192 pg/mL, whereas in combination with Exemplary Payload Neutralizing Antibody 1, free plasma MMAE Cmax reduced approximately 10-fold. Administration of a low potency control at 30 mg/kg showed less of an impact on free MMAE concentrations, aligning closer to levels observed in BV-alone group, demonstrating the importance of high affinity antibodies, such as Exemplary Payload Neutralizing Antibody 1, for payload neutralization.

Quantitative analysis of free MMAE exposure (AUC, 0-7 days) revealed significant reductions with Exemplary Payload Neutralizing Antibody 1 treatment. Free MMAE exposure decreased to 29.6% and 23.8% of BV-alone levels at 3 mg/kg and 30 mg/kg, respectively (FIG. 9B). These results underscore the potent and dose-dependent activity of exemplary payload neutralizing antibodies described herein in preventing free MMAE without off-target interference.

### Example 4: In Vivo Assessment of ADC Efficacy on PDX Tumors in Presence of Exemplary Payload Neutralizing Antibodies

The present Example demonstrates that addition of exemplary payload neutralizing antibodies can prevent and/or reduce free drug (e.g., MMAE) levels, with minimal or no impairment of ADC potency in vivo.

### Methods

These studies assessed the potential impact of reduction in free MMAE on activity of an ADC using a patient-derived xenograft (PDX) murine model of bladder cancer. In these studies, anti-Nectin4 ADC, Enfortumab vedotin, was administered alone, or in combination with an Exemplary Payload Neutralizing Antibody (1 or 2) or a non-binding isotype control.

### Tumor Growth Analysis

Female athymic nude (Crl:NU(NCr)-Foxn1^{nu}) were implanted with a PDX model ST458 subcutaneously in one flank following IACUC protocols. Animals with tumors between 200-300 mm³ were randomized and treated as outlined in Tables 7 and 8. Tumor volumes were recorded every 3-4 days, and animals were removed from the study when tumors reached 2500 mm³.

### Pharmacokinetic Analysis

To assess pharmacokinetics of Exemplary Payload Neutralizing Antibody 2, animals receiving 3 mg/kg EV in combination with 10, 3, 1, 0.3, or 0.1 mg/kg Exemplary Payload Neutralizing Antibody 2 (n=6 mice per treatment group) were divided into two cohorts (n=3 mice per cohort) for plasma sampling. Plasma samples were collected at multiple time points post-dose, and Exemplary Payload Neutralizing Antibody 2 concentrations were quantified using a qualified LC/MS/MS assay.

### Free MMAE Exposure Analysis

To assess pharmacokinetics and exposure to free MMAE, animals receiving 3 mg/kg EV alone or in combination with 0.3, 3, or 30 mg/kg Exemplary Payload Neutralizing Antibody 1 (n=12 mice per treatment group) were divided into four cohorts (n=3 mice per cohort) for plasma sampling. Plasma samples were collected at multiple time points post-dose, and free MMAE concentrations were quantified using a qualified LC/MS/MS assay. Non-compartmental analysis (NCA) was used to derive pharmacokinetic parameters for free MMAE plasma exposure (AUC). Free MMAE plasma AUCs in the presence of Exemplary Payload Neutralizing Antibody 1 were normalized to AUCs of the 3 mg/kg EV group. Animals receiving 3 mg/kg EV in combination with 10 mg/kg isotype control or 0.1, 0.3, 1, 3, or 10 mg/kg Exemplary Payload Neutralizing Antibody 2 (n=12 mice per treatment group) were divided into four cohorts (n=3 mice per cohort) for plasma sampling. Plasma samples were collected at multiple time points post-dose, and free MMAE concentrations were quantified using a qualified LC/MS/MS assay. Non-compartmental analysis (NCA) was used to derive pharmacokinetic parameters for free MMAE plasma exposure (AUC) and maximum concentrations (Cmax). Free MMAE plasma AUCs in the presence of Exemplary Payload Neutralizing Antibody 2 were normalized to AUCs of the 3 mg/kg EV+10 mg/kg isotype control group.

TABLE 7
Dosing groups for PDX model ST458, Exemplary
Payload Neutralizing Antibody 1

| | | Exemplary | Non- | | |
|---|---|---|---|---|---|
| | | Payload | binding | | |
| | Enfortumab | Neutralizing | isotype | | dose |
| Group | vedotin | Antibody 1 | control | N | schedule |
| 1 | — | — | — | 8 | NA |
| 2 | — | 30 mg/kg | — | 12 | q7dx3 |
| 3 | 1 mg/kg | — | — | 12 | q7dx3 |
| 4 | 3 mg/kg | — | — | 12 | q7dx3 |
| 5 | 3 mg/kg | 0.3 mg/kg | — | 12 | q7dx3 |
| 6 | 3 mg/kg | 3.0 mg/kg | — | 12 | q7dx3 |
| 7 | 3 mg/kg | 30.0 mg/kg | — | 12 | q7dx3 |
| 8 | 3 mg/kg | — | 30 mg/kg | 5 | q7dx3 |

TABLE 8
Dosing groups for PDX model ST458, Exemplary
Payload Neutralizing Antibody 2

| | | Exemplary | Non- | | |
|---|---|---|---|---|---|
| | | Payload | binding | | |
| | Enfortumab | Neutralizing | isotype | | dose |
| Group | vedotin | Antibody 2 | control | N | schedule |
| 1 | — | — | — | 15 | NA |
| 2 | 3 mg/kg | — | 10 mg/kg | 12 | q7dx3 |
| 3 | 3 mg/kg | 0.1 mg/kg | — | 18 | q7dx3 |
| 4 | 3 mg/kg | 1.0 mg/kg | — | 18 | q7dx3 |
| 5 | 3 mg/kg | 3.0 mg/kg | — | 18 | q7dx3 |
| 6 | 3 mg/kg | 10 mg/kg | — | 18 | q7dx3 |
| 7 | 3 mg/kg | 0.3 mg/kg | — | 18 | q7dx3 |

### Results

### Tumor Growth Analysis

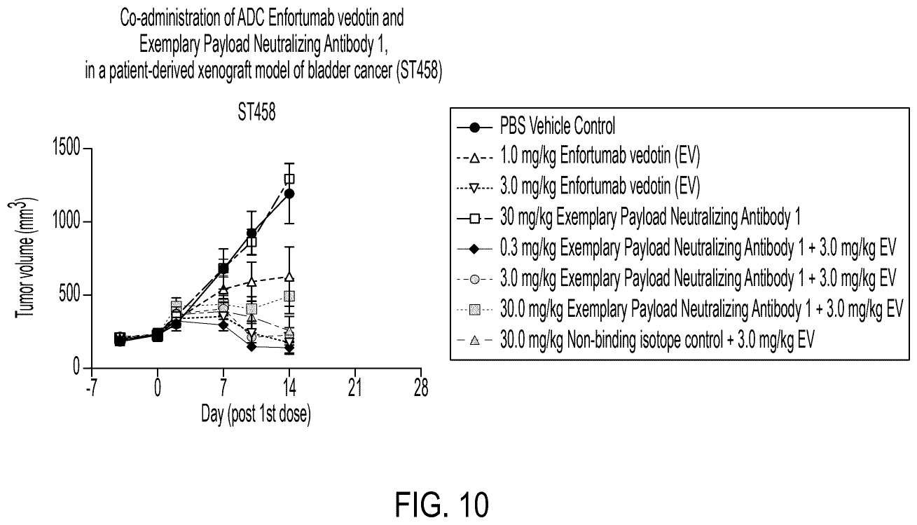

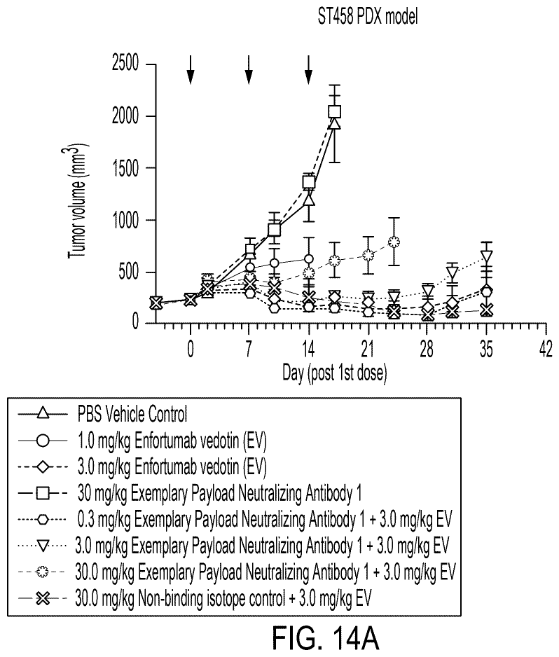

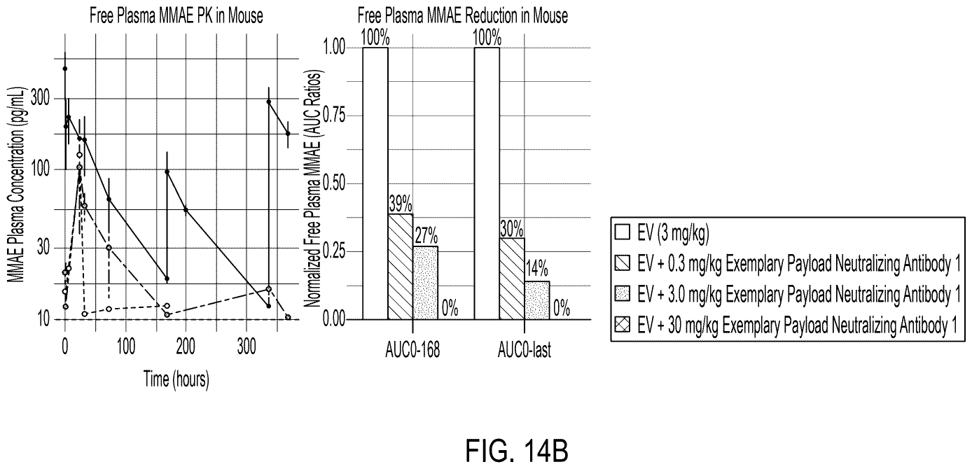

Potential impact of Exemplary Payload Neutralizing Antibodies on EV antitumor activity was assessed in the ST458 bladder cancer PDX model, which expresses moderate levels of Nectin-4 with 100% of Nectin-4-positive cells. In one experiment, activity of Enfortumab vedotin was not reduced by addition of 0.3 mg/kg or 3 mg/kg Exemplary Payload Neutralizing Antibody 1 relative to 3 mg/kg EV group, as inferred from changes in tumor volume over time. A small, qualitative reduction in efficacy was observed in the presence of 30 mg/kg Exemplary Payload Neutralizing Antibody 1 on day 14 post-1^{st} dose; however, this difference was not significant using a 2-way ANOVA with Dunnett's post hoc multiple-comparisons test (FIG. 10, FIG. 14A). Starting after day 14, the 3 mg/kg EV +30.0 mg/kg Exemplary Payload Neutralizing Antibody 1 group (Group 7) was found to have a tumor volume statistically larger than the 3 mg/kg EV group (Group 4) (FIG. 14A).

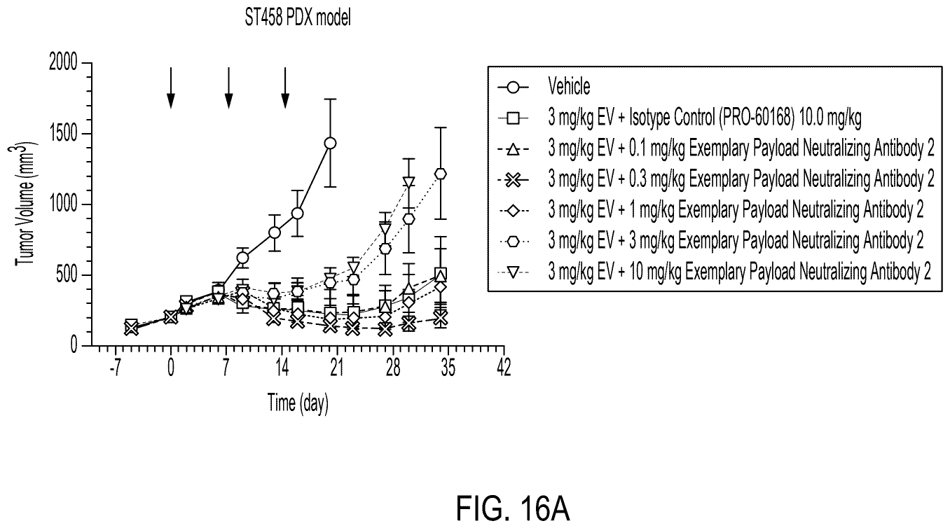

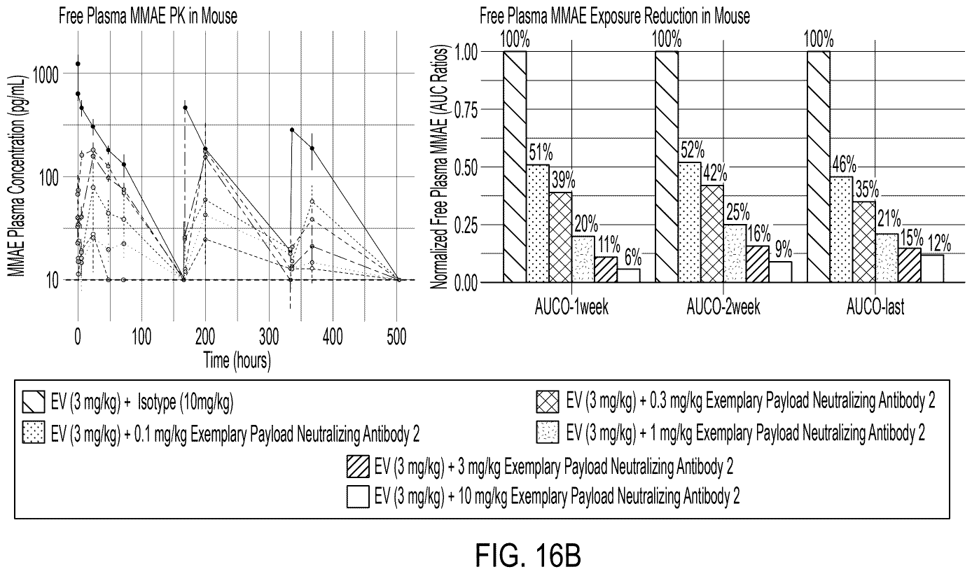

Similarly, potential impact of Exemplary Payload Neutralizing Antibody 2 on EV antitumor activity was assessed in the ST458 bladder cancer PDX model. Co-administration of Exemplary Payload Neutralizing Antibody 2 at 0.1, 0.3, or 1 mg/kg did not alter EV efficacy. As shown in FIG. 16A, at these lower Exemplary Payload Neutralizing Antibody 2 doses, tumor volumes remained comparable to that observed with 3 mg/kg EV combined with 10 mg/kg isotype control using a 2-way ANOVA with Dunnett's post hoc multiple-comparisons test.

In contrast, co-administration of Exemplary Payload Neutralizing Antibody 2 at 3 and 10 mg/kg reduced EV efficacy. As shown in FIG. 16A, Tumor volumes in these groups were statistically significantly larger than those in the 3 mg/kg EV+10 mg/kg isotype control group starting after day 27.

### Pharmacokinetic Analysis

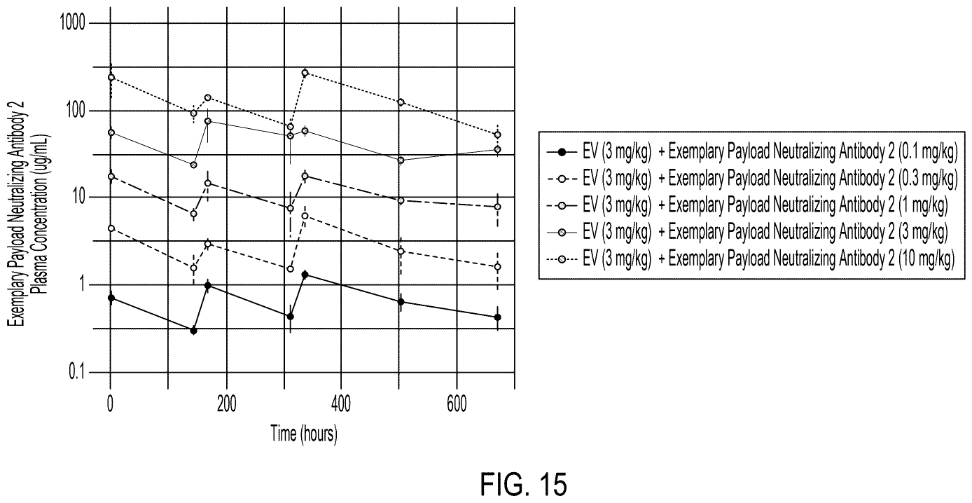

Pharmacokinetics of Exemplary Payload Neutralizing Antibody 2 were examined in tumor-bearing mice, which were co--administered 3 mg/kg EV and Exemplary Payload Neutralizing Antibody 2 IV once every 7 days for 3 doses. Exemplary Payload Neutralizing Antibody 2 was evaluated in mouse plasma across five dose levels (0.1, 0.3, 1, 3, and 10 mg/kg). As shown in FIG. 15, the PK profile of Exemplary Payload Neutralizing Antibody 2 was consistent with expectations for this modality, demonstrating linear and dose-proportional exposure across the evaluated dose range

### Free MMAE Exposure Analysis

Free MMAE was assessed in tumor-bearing mice co-administered 3 mg/kg EV and Exemplary Payload Neutralizing Antibody 1. Mouse free plasma MMAE concentrations were measured over time (FIG. 14B, Left Panel) and free plasma MMAE AUC normalized to the 3 mg/kg EV alone group (FIG. 14B, Right Panel). As shown in FIG. 14, the normalized free plasma MMAE AUCs over a period of 1-week (AUC₀₋₁₆₈ₕ) at Exemplary Payload Neutralizing Antibody 1 doses of 0.3 mg/kg (Group 5), 3 mg/kg (Group 6), and 30 mg/kg (Group 7) were 39%, 27%, and 0%, (corresponding to free MMAE exposure reductions of 61%, 73%, and 100%), respectively. Over the entire PK sampling duration of 368 hours (AUC₀₋ₗₐₛₜ), the normalized free plasma MMAE exposures were 30%, 14%, and 0%, (corresponding to free MMAE exposure reductions of 70%, 86%, and 100%), respectively. The 0% AUC value reported for the 30 mg/kg group (Group 7) indicates that free MMAE levels were below the limit of quantification (LOQ) throughout the time period.Free MMAE was also assessed in tumor-bearing mice co-administered 3 mg/kg EV and Exemplary Payload Neutralizing Antibody 2. Co-administration of Exemplary Payload Neutralizing Antibody 2 at 0.1, 0.3, and 1 mg/kg reduced free plasma MMAE exposures. As shown in FIG. 16B, free plasma MMAE AUCs over the first 168 hours (AUC_{0-1week}) for 0.1, 0.3, and 1 mg/kg Exemplary Payload Neutralizing Antibody 2 were reduced to 51%, 39%, and 20% of control, corresponding to exposure reductions of 49%, 61%, and 80%, respectively. Over a period of the first 336 hours (AUC_{0-2week}), normalized free plasma MMAE exposures were 52%, 42%, and 25%, which corresponds to a free MMAE exposure reduction of 48%, 58%, and 75%, respectively. Over the entire PK sampling duration of 672 hours (AUC₀₋ₗₐₛₜ), the normalized free plasma MMAE exposures were 46%, 35%, and 21%, which corresponds to a free MMAE exposure reduction of 54%, 65%, and 79%, respectively. Normalized free plasma MMAE Cmax values for these doses were 14%, 13%, and 6%, corresponding to reductions of 86%, 87%, and 94%, respectively. Co-administration of Exemplary Payload Neutralizing Antibody 2 at 3 and 10 mg/kg markedly decreased free plasma MMAE exposure. As shown in FIG. 16B, at 3 and 10 mg/kg Exemplary Payload Neutralizing Antibody 2, normalized free plasma MMAE AUCs over one week (168 hours) (AUC_{0-1week}) were reduced to 11% and 6% of control, corresponding to 89% and 94% reductions, respectively. Over a period of 2 weeks (336 hours) (AUC_{0-2week}), normalized free plasma MMAE exposures were 16%, and 9%, which corresponds to a free MMAE exposure reduction of 84%, and 91%, respectively. Over the entire PK sampling duration of 672 hours (AUC₀₋ₗₐₛₜ), normalized free plasma MMAE exposures were 15%, and 12%, which corresponds to a free MMAE exposure reduction of 85%, and 88%, respectively. Cmax values at these higher doses were reduced to 3% of control, corresponding to 97% reductions.

These findings demonstrate that reductions in free MMAE of up to 73% and 86% for AUC₀₋₁₆₈ₕ and AUC₀₋ₗₐₛₜ (Exemplary Payload Neutralizing Antibody 1, 3 mg/kg dose level; Group 6), respectively, do not impair the pharmacological activity of an MMAE-based ADC. In addition, these findings demonstrate that reductions in free MMAE plasma exposure up to 80%, 75%, and 94% for AUC_{0-1week}, AUC_{0-2week}, and Cmax (Exemplary Payload Neutralizing Antibody 2, 1 mg/kg dose level), respectively, did not impact the antitumor efficacy of EV. In contrast, reductions in free MMAE plasma exposure of at least 89%, 84%, and 97%, for AUC_{0-1week}, AUC_{0-2week}, and Cmax (Exemplary Payload Neutralizing Antibody 2, 3 mg/kg dose level), respectively, had a negative impact on the antitumor efficacy of EV.

### Example 5: In Vivo Assessment of ADC Efficacy and Toxicity in Cynomolgus in Presence of Exemplary Payload Neutralizing Antibodies

The present example assesses the potential toxicities (i.e., neutropenia and skin toxicity) of Enfortumab Vedotin (EV) when administered intravenously (IV bolus) in cynomolgus monkeys, with or without prior intravenous administration of an Exemplary Payload Neutralizing Antibody.

### Methods

EV was administered via IV bolus in cynomolgus monkeys at 4 mg/kg, with or without prior IV bolus administration of Exemplary Payload Neutralizing Antibody 2, an MMAE Neutralizing Antibody, at low, mid or high doses (single dose) or once weekly dosing for 4 weeks. Skin abnormalities, body weights, organ weights, bone marrow cellularity, macroscopic pathology, and hematology (e.g., neutropenia) were analyzed. In addition, the toxicokinetic (TK) characteristics of Enfortumab Vedotin, Exemplary Payload Neutralizing Antibodies, and free-MMAE in serum were assessed.

### Results

Enfortumab Vedotin-related skin abnormalities of erythema, dryness, scaling, flaking and/or lacerations/abscesses were noted in cynomolgus monkeys treated with EV alone, necessitating various veterinary treatments. When administered in combination with increasing dose levels of Exemplary Payload Neutralizing Antibody 2, the severity and incidence of Enfortumab Vedotin-related skin abnormalities were lessened in a dose-dependent manner.

Neutropenia was assayed in cynomolgus monkeys treated with EV alone or in combination with Exemplary Payload Neutralizing Antibody 2. Results are shown in Table 9. In Table 9, Grade 3+neutropenia is defined as ANC<1×10³ cells/μL, grade 4 neutropenia is defined as ANC<0.5×10³ cells/μL, time-to-neutropenia is defined as the time from dose administration to the first occurrence of neutropenia reported as median (min-max) days

TABLE 9
Neutropenia in cynomolgus monkeys treated with EV alone or in combination
with Exemplary Payload Neutralizing Antibody 2

| | | | | Time-to- | Time-to- |
|---|---|---|---|---|---|
| | | Grade 3+ | Grade 4 | Neutropenia | Neutropenia |
| Group | Treatment | Neutropenia | Neutropenia | Grade 3+ | Grade 4+ |
| 2 | 4 mg/kg EV | 3/3 | 3/3 | 8 (6-8) | 8 (6-9) |
| 3 | 4 mg/kg EV + | 2/2 | 2/2 | 7.5(7-8) | 8.5 (8-9) |
| | Exemplary Payload | | | | |
| | Neutralizing | | | | |
| | Antibody 2, low dose | | | | |
| 4 | 4 mg/kg EV+ | 2/2 | 1/2 | 12 (10-14) | 14(14-14) |
| | Exemplary Payload | | | | |
| | Neutralizing | | | | |
| | Antibody 2, mid dose | | | | |
| 5 | 4 mg/kg EV + | 1/3 | 1/3 | 14(14-14) | 14(14-14) |
| | Exemplary Payload | | | | |
| | Neutralizing | | | | |
| | Antibody 2, high | | | | |
| | dose | | | | |

As shown in Table 9, at mid and high doses of Exemplary Payload Neutralizing Antibody, Enfortumab Vedotin-related changes in hematology parameters, including neutropenia, and microscopic changes in the bone marrow were generally less pronounced or absent. No Enfortumab Vedotin-related changes were noted in any parameter evaluated when administered in combination with once weekly doses of Exemplary Payload Neutralizing Antibody 2 for 4 weeks.

These data indicate that reductions of approximately 50% in free circulating MMAE exposure are sufficient to mitigate MMAE-related toxicities (e.g., neutropenia), providing at least one mechanistic rationale for the use of Payload Neutralizing Antibodies described herein to enhance the therapeutic index of MMAE-based ADC therapies.

### EQUIVALENTS

It is to be appreciated by those skilled in the art that various alterations, modifications, and improvements to various aspects of innovations described herein will readily occur to those skilled in the art. Such alterations, modifications, and improvements are intended to be part of various aspects of innovations described herein and are intended to be within the spirit and scope of the such aspects of innovation. Accordingly, the foregoing description and drawing are by way of example only and any various aspects of innovations described herein if further described in detail by the claims that follow.

Those skilled in the art will appreciate typical standards of deviation or error attributable to values obtained in assays or other processes described herein. The publications, websites and other reference materials referenced herein to describe the background of various aspects of innovations described herein and to provide additional detail regarding its practice are hereby incorporated by reference in their entireties.

1. An antibody or antigen-binding fragment thereof capable of binding to a free drug of an antibody drug conjugate (ADC), comprising:
(a) a heavy chain variable region (VH) comprising a VHCDR1 amino acid sequence of SEQ ID NO: 18; a VHCDR2 amino acid sequence of SEQ ID NO: 19; and a VHCDR3 amino acid sequence of SEQ ID NO: 20; and a light chain variable region (VL) comprising a VLCDR1 amino acid sequence of SEQ ID NO: 28, a VLCDR2 amino acid sequence of SEQ ID NO: 29, and a VLCDR3 amino acid sequence of SEQ ID NO: 30;
(b) a VH comprising a VHCDR1 amino acid sequence of SEQ ID NO: 21; a VHCDR2 amino acid sequence of SEQ ID NO: 22; and a VHCDR3 amino acid sequence of SEQ ID NO: 23; and a VL comprising a VLCDR1 amino acid sequence of SEQ ID NO: 31, a VLCDR2 amino acid sequence of SEQ ID NO: 32, and a VLCDR3 amino acid sequence of SEQ ID NO: 30;
(c) a VH comprising a VHCDR1 amino acid sequence of SEQ ID NO: 24; a VHCDR2 amino acid sequence of SEQ ID NO: 25; and a VHCDR3 amino acid sequence of SEQ ID NO: 23; and a VL comprising a VLCDR1 amino acid sequence of SEQ ID NO: 31, a VLCDR2 amino acid sequence of SEQ ID NO: 32, and a VLCDR3 amino acid sequence of SEQ ID NO: 30;
(d) a VH comprising a VHCDR1 amino acid sequence of SEQ ID NO: 1; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 11, a VLCDR2 amino acid sequence of SEQ ID NO: 12, and a VLCDR3 amino acid sequence of SEQ ID NO: 13;
(e) a VH comprising a VHCDR1 amino acid sequence of SEQ ID NO: 4; a VHCDR2 amino acid sequence of SEQ ID NO: 5; and a VHCDR3 amino acid sequence of SEQ ID NO: 6; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 13;
(f) a VH comprising a VHCDR1 amino acid sequence of SEQ ID NO: 7; a VHCDR2 amino acid sequence of SEQ ID NO: 8; and a VHCDR3 amino acid sequence of SEQ ID NO: 6; and a VL comprising a VLCDR1 amino acid sequence of SEQ ID NO: 14, a VLCDR2 amino acid sequence of SEQ ID NO: 15, and a VLCDR3 amino acid sequence of SEQ ID NO: 13;
(g) a VH comprising a VHCDR1 amino acid sequence of SEQ ID NO: 35; a VHCDR2 amino acid sequence of SEQ ID NO: 36; and a VHCDR3 amino acid sequence of SEQ ID NO: 37; and a VL comprising a VLCDR1 amino acid sequence of SEQ ID NO: 44, a VLCDR2 amino acid sequence of SEQ ID NO: 45, and a VLCDR3 amino acid sequence of SEQ ID NO: 46;
(h) a VH comprising a VHCDR1 amino acid sequence of SEQ ID NO: 35; a VHCDR2 amino acid sequence of SEQ ID NO: 38; and a VHCDR3 amino acid sequence of SEQ ID NO: 39; and a VL comprising a VLCDR1 amino acid sequence of SEQ ID NO: 47, a VLCDR2 amino acid sequence of SEQ ID NO: 48, and a VLCDR3 amino acid sequence of SEQ ID NO: 46; or
(i) a VH comprising a VHCDR1 amino acid sequence of SEQ ID NO: 40; a VHCDR2 amino acid sequence of SEQ ID NO: 41; and a VHCDR3 amino acid sequence of SEQ ID NO: 39; and a VL comprising a VLCDR1 amino acid sequence of SEQ ID NO: 47, a VLCDR2 amino acid sequence of SEQ ID NO: 48, and a VLCDR3 amino acid sequence of SEQ ID NO: 46.

2. An antibody or antigen-binding fragment thereof capable of binding to a free drug of an antibody drug conjugate (ADC), comprising:
(a) a VH comprising an amino acid sequence having at least about 85% sequence identity to an amino acid sequence of any one of SEQ ID NOs: 9, 26, or 42; and
(b) a VL comprising an amino acid sequence having at least about 85% sequence identity to an amino acid sequence of any one of SEQ ID NOs: 16, 33, or 49.

3. The antibody or antigen-binding fragment thereof of claim 2, comprising:
(a) a VH comprising an amino acid sequence of any one of SEQ ID NOs: 9, 26, or 42; and
(b) a VL comprising an amino acid sequence of any one of SEQ ID NOs: 16, 33, or 49.

4. The antibody or antigen-binding fragment thereof of claim 3, comprising:
(a) a VH comprising the amino acid sequence of SEQ ID NO: 9 and a VL comprising the amino acid sequence of SEQ ID NO: 16;
(b) a VH comprising the amino acid sequence of SEQ ID NO: 26 and a VL comprising the amino acid sequence of SEQ ID NO: 33; or
(c) a VH comprising the amino acid sequence of SEQ ID NO: 42 and a VL comprising the amino acid sequence of SEQ ID NO: 49.

5. The antibody or antigen-binding fragment thereof of claim 1, wherein said antibody or antigen-binding fragment thereof is:
(i) a humanized antibody or antigen-binding fragment thereof, and/or
(ii) a monospecific antibody or antigen-binding fragment thereof, or a bispecific antibody or antigen-binding fragment thereof.

6. The antibody or antigen-binding fragment thereof of claim 1, comprising a Fab, F(ab′)₂, Fv, or a single chain Fv fragment (scFv).

7. The antibody or antigen-binding fragment thereof of claim 1, comprising:
(i) a heavy chain constant region selected from IgG1, IgG2, IgG3, and IgG4, and/or
(ii) a light chain constant region chosen from kappa or lambda.

8. The antibody or antigen-binding fragment thereof of claim 1, comprising:
(a) a heavy chain comprising an amino acid sequence with at least about 80% sequence identity to an amino acid sequence of any one of SEQ ID NOs: 10, 27, or 43; and
(b) a light chain comprising an amino acid sequence with at least about 80% sequence identity to an amino acid sequence of any one of SEQ ID NOs: 17, 34, or 50.

9. The antibody or antigen-binding fragment thereof of claim 8, comprising:
(a) a heavy chain comprising an amino acid sequence of any one of SEQ ID NOs: 10, 27, or 43; and
(b) a light chain comprising an amino acid sequence of any one of SEQ ID NOs: 17, 34, or 50.

10. The antibody or antigen-binding fragment thereof of claim 9, comprising:
(a) a heavy chain comprising the amino acid sequence of SEQ ID NO: 10 and a light chain comprising the amino acid sequence of SEQ ID NO: 17;
(b) a heavy chain comprising the amino acid sequence of SEQ ID NO: 27 and a light chain comprising the amino acid sequence of SEQ ID NO: 34; or
(c) a heavy chain comprising the amino acid sequence of SEQ ID NO: 43 and a light chain comprising the amino acid sequence of SEQ ID NO: 50.

11. The antibody or antigen-binding fragment thereof of claim 1, wherein the drug is an auristatin.

12. The antibody or antigen-binding fragment thereof of claim 11, wherein the auristatin is monomethyl auristatin E (MMAE), monomethyl auristatin F (MMAF), or monomethyl auristatin D (MMAD).

13. A pharmaceutical composition comprising the antibody or antigen-binding fragment thereof of claim 1, and a pharmaceutically acceptable carrier.

14. A polynucleotide encoding the antibody or antigen-binding fragment thereof of claim 1.

15. An expression vector comprising the polynucleotide of claim 14.

16. A host cell comprising the expression vector of claim 15.

17. A method of making an antibody or antigen-binding fragment thereof, comprising culturing the host cell of claim 16 under conditions suitable for gene expression.

18. A method of treating or preventing off-target effects of a free drug in a subject comprising administering a therapeutically effective amount of the pharmaceutical composition of claim 13 to the subject.

19. (canceled)

20. A method of treating a subject having a disease, disorder, or condition comprising administering a therapeutically effective amount of the pharmaceutical composition of claim 13 to the subject.

21. The method of claim 18, wherein the subject has or is at risk of developing a cancer.

22.-24. (canceled)
